(19) United States
(12) Patent Application Publication
(10) Pub. No.: US 2011/0003849 A1
(43) Pub. Date: Jan. 6, 2011

(54) SOLUBLE EPOXIDE HYDROLASE INHIBITORS, COMPOSITIONS CONTAINING SUCH COMPOUNDS AND METHODS OF TREATMENT

(76) Inventors: Hong Shen, West Windsor, NJ (US); Fa-Xiang Ding, Staten Island, NY (US); Steven L. Colletti, Princeton Junction, NJ (US)

Correspondence Address: MERCK, P O BOX 2000, RAHWAY, NJ, 07065-0907, US

(21) Appl. No.: 12/919,323

(22) PCT Filed: Feb. 24, 2009
(86) PCT No.: PCT/US09/34939

§ 371 Date: Aug. 25, 2010

Related U.S. Application Data
(60) Provisional application No. 61/068,094, filed on Mar. 4, 2008.

(51) Int. Cl. A61K 31/439; C07D 451/02; C07D 453/02; C07D 487/08; A61K 31/4439; A61P 3/10

(52) U.S. Cl. 514/304; 546/125; 546/133; 514/305; 546/276.7; 514/339

(57) ABSTRACT

Compounds of the formula: as well as pharmaceutically acceptable salts and hydrates thereof, that are useful for treating diabetes, inflammation, atherosclerosis, hypertension, pain and the like are disclosed. Pharmaceutical compositions and methods of use are also included.

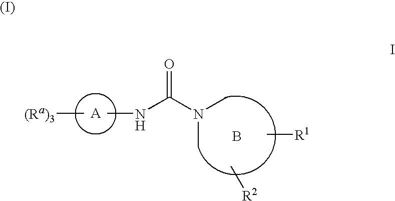

### BACKGROUND OF THE INVENTION

The present invention relates to bridged bicyclic amine-derived trisubstituted urea compounds possessing soluble epoxide hydrolase (sEH) inhibitory activity, compositions containing sEH inhibitory compounds, and methods of treatment relating to diseases and conditions in which soluble epoxide hydrolase is implicated.

Epoxide hydrolases are a group of enzymes ubiquitous in nature, detected in species ranging from plants to mammals. These enzymes are functionally related in that they catalyze the addition of water to an epoxide, resulting in the formation of a diol. Diols are frequently found as intermediates in metabolic pathways.

Several types of epoxide hydrolases have been characterized, including soluble epoxide hydrolase, also referred to as cytosolic epoxide hydrolase, cholesterol epoxide hydrolase, LTA4 hydrolase, hepoxilin hydrolase, and microsomal epoxide hydrolase (mEH), (Fretland, et al. Chem. Biological Interactions, 129: 41-59 (2000)). Epoxide hydrolases have been found in mammalian heart, kidney and liver tissue (Vogel et al. Eur. J. Biochem. 126: 425-431 (1982) Schladt et al., Biochem Pharmacol. 35: 3309-3316 (1986). Epoxide hydrolases have also been detected in human blood components including lymphocytes (e.g., T-lymphocytes), monocytes, erythrocytes, and platelets. In the blood, most of the sEH detected was present in lymphocytes (Seidegard, et al. Cancer Research 44: 3654-3660 (1984).

The epoxide hydrolases differ in their specificity towards epoxide substrates. For example, sEH is selective for aliphatic epoxides such as epoxide fatty acids while microsomal epoxide hydrolase (mEH) is more selective for cyclic and arene epoxides. The primary known physiological substrates of sEH are the four regioisomeric epoxides of arachidonic acid, 5,6-, 8,9-, 11,12- and 14,15-epoxyeicosatrienoic acid, also known as epoxyeicosatrienoic acids or EETs. It has been reported that red blood cells can be reservoirs of EETs as well (Mini review: Jiang, H. Prostaglandins & other Lipid Mediators 2007, 82, 4). Also known to be substrates for sEH are epoxides of linoleic acid known as leukotoxin or isoleukotoxin.

The EETs are known to be vasodilatory mediators. Their role in vessel relaxation of peripheral vessels and renal microvessels, stems from their activation of Ca(+2)-activated potassium BK(Ca) ion channels. Furthermore 11,12-EET has been identified as the endothelial derived hyperpolarization factor (EDHF). These properties of EETs render them an attractive target for elevation in vivo, with application to improving endothelial dysfunction. Endothelial dysfunction plays a significant role in a large number of pathological conditions including type 2 diabetes, insulin resistance, hypertension, atherosclerosis, coronary artery disease, angina, ischemia, ischemic stroke, Raynaud's disease and renal disease (Cersosimo, et.al. Diabetes/Metabolism Research and Reviews 2006, 22, 423). Endothelial mediated vessel relaxation can contribute 25-40% of insulin stimulated glucose uptake during a euglycemic clamp (Kim, et.al. Circulation 2006, 113, 1888). Hence, one object of the present invention is to provide compounds that are useful for the treatment of type 2 diabetes and related conditions.

Other effects of EET's involve kidney function. In angiotensin II infused rats, treatment with a selective sEH inhibitor attenuated the afferent arteriolar diameter in the kidney and lowered urinary albumin secretion, a marker of compromised renal function, suggesting antihypertensive and renal vascular protective effects of increased EET levels. Administration of a (selective) sEH inhibitor to angiotensin II treated rats was demonstrated to lower systolic blood pressure (Imig, et al. Hypertension, 39: 690-694 (2002)). Hence, one object of the present invention is to provide end organ protection along with the treatment of hypertension.

EET's, and especially 11,12-EET, also have been shown to exhibit anti-inflammatory properties (Node, et al. Science 285: 1276-1279 (1999)); Campbell, TIPS 21: 125-127 (2000); Zeldin et al. TIPS 21: 127-128 (2000)). Node et al. demonstrated that 11,12-EET decreased expression of cytokine induced endothelial cell adhesion molecules, especially VCAM-1. Moreover, EETs prevented leukocyte adhesion to the vascular wall and the mechanism responsible involved inhibition of NFκB and IKB kinase. Vascular inflammation plays a role in endothelial dysfunction (Kessler, et al. Circulation, 99: 1878-1884 (1999)). Hence, the ability of EETs to inhibit the NFκB pathway should also help ameliorate this condition. In addition, the administration of EETs and/or the administration of a selective sEH inhibitor was demonstrated to attenuate tobacco smoke induced inflammation, as assessed by total bronchoalveolar lavage cell numbers and concomitant reduction in neutrophils, alveolar macrophages and lymphocytes.

Hammock et al. have demonstrated usefulness in the treatment of inflammatory diseases, in particular, adult respiratory distress syndrome and other acute inflammatory conditions mediated by lipid metabolites, by the administration of inhibitors of epoxide hydrolase (WO98/06261, U.S. Pat. No. 5,955,496).

More recently, Hammock, et al. disclosed certain biologically stable inhibitors of sEH for the treatment of inflammatory diseases, for use in affinity separations of epoxide hydrolases and in agricultural applications (U.S. Pat. No. 6,150,415). Hammock et al. generally described compounds that can be used to deliver a reactive functionality to the catalytic site, e.g., alkylating agents or Michael acceptors, and that these reactive functionalities can be used to deliver fluorescent or affinity labels to the enzymes active site for enzyme detection. Certain urea and carbamate inhibitors of sEH have also been described in the literature (Morisseau, et al. Proc. Nat. Acad. Sci. 96: 8849-8854 (1999)).

A number of other chemical classes of sEH inhibitors have been identified. Among these are chalcone oxide derivatives (Miyamoto, et al. Arch. Biochem. Biophys. 254: 203-213 (1987)) and various trans-3-phenylglyucidols (Dietze, et al. Biochem. Pharm. 42: 1163-1175 (1991)) and Dietze, et al. Comp. Biochem. Physiol. B. 104: 309-314 (1993)).

It has recently been shown that sEH inhibition reduces COX-2 expression in mammals, and decreases PGE2 and PGD2 levels, similar to coxibs. Therefore, sEH inhibitors could be indicated for inflammatory pain (Schmelzer, et.al. PNAS 2006, 103, 13646). It has also been disclosed that 14,15-EET is 100-fold more potent than morphine dosed vPAG in rat brains, and EETs induce Met-enkephalin release in the spinal cord. This suggests that sEH inhibitors could also be used for CNS analgesia (Harder, D. presented at 9^{th} Annual WEC, March 2007).

The anti-inflammatory functions of EETs also indicate that it is possible to use sEH inhibitors as ophthalmic agents to alleviate eye disorders, such as reducing intraocular pressure and reducing progression of age-related macular degeneration (WO 2007/009001 A1).

All four EET regioisomers inhibit arachidonic acid-induced aggregation of human platelets, induce t-PA expression and hyperpolarize platelets (Node, et al. T. Biol. Chem. 2001, 276(19), 15983). This supports the potential use of sEH inhibitors as anti-thrombotic agents.

An object of the present invention is to provide compounds that are useful for the treatment of hyperlipidemias, dyslipidemias, atherosclerosis and related conditions.

Another object is to provide a pharmaceutical composition for oral use.

These and other objects will be apparent from the description provided herein.

### SUMMARY OF THE INVENTION

A compound represented by formula I:

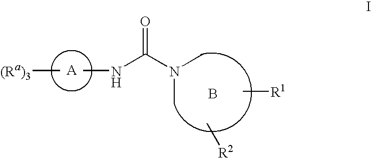

or a pharmaceutically acceptable salt or solvate thereof wherein:

ring A represents Aryl, HAR, Hetcy, C₃₋₇cycloalkyl, C₅₋₇cycloalkyl fused to an Aryl or HAR group, Aryl or HAR fused to C₅₋₇cycloalkyl, or C₆₋₁₀bicycloalkyl;

ring B represents a bridged bicyclic heterocyclic group having 1 nitrogen atom, 0-1 oxygen atom and 7-9 total atoms;

each R^{a} is defined as follows:

a) each R^{a} is H or halo, or

b) 1-2 R^{a} groups represent H or halo,
0-1 R^{a} represents Aryl, HAR or Hetcy, each of which being optionally substituted with 1-3 halo, C₁₋₃alkyl, haloC₁₋₃alkyl, OC₁₋₃alkyl or OhaloC₁₋₃alkyl groups, and 0-1 —CO₂R^{b} group;
and any remaining R^{a} groups are selected from the group consisting of: C₁₋₃alkyl, OC₁₋₃alkyl, haloC₁₋₃alkyl, OhaloCₜ₋₃alkyl, S(O)ₓC₁₋₃alkyl, S(O)ₓ-haloC₁₋₃alkyl, S(O)ₓAryl wherein x is 0, 1 or 2, CO₂R^{b} or C₁₋₃alkyl-CO₂R^{b}, wherein R^{b} is H, C₁₋₄alkyl, haloC₁₋₄alkyl, Aryl, HAR or Hetcy;

R¹ is selected from the group consisting of: H, halo, C₁₋₃alkyl and haloC₁₋₃alkyl;

and R² is selected from Aryl(R^{X})ₚ and HAR(R^{x})_{q}, wherein p represents an integer of 1-5, q represents an integer of 1-4, each R^{x} is H, or 1-2 R^{x} groups are selected from the group consisting of: halo; C₁₋₅alkyl(R^{b})₃; OC₁₋₃alkyl(R^{b})₃; S(O)ₓC₁₋₃alkyl(R^{b})₃; S(O)ₓAryl; NH₂; NH(C₁₋₄alkyl(R^{b})₃); N(C₁₋₄alkyl(R^{b})₃)₂; CO₂R^{b}; Aryl, HAR and Hetcy, wherein said Aryl, HAR and Hetcy are each optionally substituted with 1-3 halo, C₁₋₃alkyl, haloC₁₋₃alkyl, OC₁₋₃alkyl or OhaloC₁₋₃alkyl groups, and 0-1 CO₂—C₁₋₆alkyl groups, and any remaining R^{x} groups are H, halo, C₁₋₃alkyl or haloC₁₋₃alkyl.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl, cyclopropyl and the like. Cycloalkyl is thus a subset of alkyl; if no number of atoms is specified, 3-7 carbon atoms are intended, forming 1-3 carbocyclic rings that are fused. “Cycloalkyl” can also be fused to an aryl or heteroaryl group. Examples of cycloalkyl include cyclopropyl, cyclobutyl, cyclopentyl, cyclohexyl, cycloheptyl, tetrahydronaphthyl, decahydronaphthyl, indanyl and the like. Haloalkoxy, Ohaloalkyl and haloOalkyl are used interchangeably and refer to halo substituted alkyoxy groups linked through the oxygen atom. Haloalkyl and haloalkoxy include mono-substituted as well as multiple substituted alkyl and alkoxy groups, up to perhalo substituted alkyl and alkoxy. For example, trifluoromethyl and trifluoromethoxy are included.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means mono-, bicyclic and tricyclic aromatic ring systems containing at least one heteroatom selected from O, S, S(O), SO₂ and N, with each ring containing 5 to 6 atoms. HAR groups may contain from 5-14, preferably 5-13 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl, quinolyl, isoquinolyl, indolyl, dihydroindolyl, quinoxalinyl, quinazolinyl, naphthyridinyl, pteridinyl, 2,3-dihydrofuro(2,3-b)pyridyl and the like. Heteroaryl also includes aromatic carbocyclic or heterocyclic groups fused to heterocycles that are non-aromatic or partially aromatic, and optionally containing a carbonyl. Examples of additional heteroaryl groups include indolinyl, dihydrobenzofuranyl, dihydrobenzothiophenyl, dihydrobenzoxazolyl, and aromatic heterocyclic groups fused to cycloalkyl rings.

Heteroaryl also includes such groups in charged form, e.g., pyridinium.

“Heterocyclyl” (Hetcy) unless otherwise specified, means mono- and bicyclic saturated and partially saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, tetrahydrofuranyl, 1,4-dioxanyl, morpholinyl, thiomorpholinyl, tetrahydrothienyl and the like. Heterocycles can also exist in tautomeric forms, e.g., 2- and 4-pyridones. Heterocycles moreover includes such moieties in charged fowl, e.g., piperidinium.

The term “bridged bicyclic heterocyclic group” refers to the ring designated B in Formula I and is a bridged two ring moiety having 7-9 atoms, one of which is a nitrogen atom and 0-1 of which is an oxygen atom. The bridge can be one to three atoms, and can contain carbon, oxygen or nitrogen. Examples of preferred bridged heterocycles are as follows:

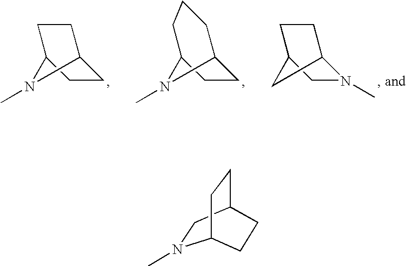

“Halogen” (Halo) includes fluorine, chlorine, bromine and iodine.

In its broadest aspect, the invention relates to compounds represented by formula I:

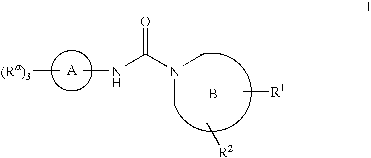

or a pharmaceutically acceptable salt or solvate thereof wherein:

ring A represents Aryl, HAR, Hetcy, C₃₋₇cycloalkyl, C₅₋₇cycloalkyl fused to an Aryl or HAR group, Aryl or HAR fused to C₅₋₇cycloalkyl, or C₆₋₁₀bicycloalkyl;

ring B represents a bridged bicyclic heterocyclic group having 1 nitrogen atom, 0-1 oxygen atom and 7-9 total atoms;

each R^{a} is defined as follows:

a) each R^{a} is H or halo, or

b) 1-2 R^{a} groups represent H or halo,
0-1 R^{a} represents Aryl, HAR or Hetcy, each of which being optionally substituted with 1-3 halo, C₁₋₃allyl, haloC₁₋₃alkyl, OC₁₋₃alkyl or OhaloC₁₋₃alkyl groups, and 0-1 —CO₂R^{b} group;
and any remaining R^{a} groups are selected from the group consisting of C₁₋₃alkyl, OC₁₋₃alkyl, haloC₁₋₃alkyl, OhaloC₁₋₃alkyl, S(O)ₓC₁₋₃alkyl, S(O)ₓ-haloC₁₋₃alkyl, S(O)ₓAryl wherein x is 0, 1 or 2, CO₂R^{b} or C₁₋₃alkyl-CO₂R^{b}, wherein R^{b} is H, haloC₁₋₄alkyl, Aryl, HAR or Hetcy;

R¹ is selected from the group consisting of: H, halo, C₁₋₃alkyl and haloC₁₋₃alkyl;

and R² is selected from Aryl(R^{x})ₚ and HAR(R^{x})_{q}, wherein p represents an integer of 1-5, q represents an integer of 1-4, each R^{x} is H, or 1-2 R^{x} groups are selected from the group consisting of: halo; C₁₋₅alkyl(R^{b})₃; OC₁₋₃alkyl(R^{b})₃; S(O)ₓC₁₋₃alkyl(R^{b})₃; S(O)ₓAryl; NH₂; NH(C₁₋₄alkyl(R^{b})₃); N(C₁₋₄alkyl(R^{b})₃)₂; CO₂R^{b}; Aryl, HAR and Hetcy, wherein said Aryl, HAR and Hetcy are each optionally substituted with 1-3 halo, C₁₋₃alkyl, haloC₁₋₃alkyl, OC₁₋₃alkyl or OhaloC₁₋₃alkyl groups, and 0-1 CO₂—C₁₋₆alkyl groups, and any remaining R^{x} groups are H, halo, C₁₋₃alkyl or haloC₁₋₃alkyl.

A subset of compounds that is of interest is described with respect to formula I wherein R^{a} is selected from the group consisting of H, F, Cl, C₁₋₃alkyl, OC₁₋₃alkyl, haloC₁₋₃alkyl, OhaloC₁₋₃alkyl, and Aryl, HAR or Hetcy, each of which is optionally substituted with 1-3 halo, haloC₁₋₃alkyl, OC₁₋₃alkyl or OhaloC₁₋₃alkyl groups, and 0-1 —CO₂R^{b} group.

Another subset of compounds that is of interest is described with respect to formula I wherein R^{a} is selected from the group consisting of H, Cl, F, CH₃, CF₃, OCF₃ and Aryl that is optionally substituted with 1-3 halo, C₁₋₃alkyl, haloC₁₋₃alkyl, OC₁₋₃alkyl or OhaloC₁₋₃alkyl groups, and 0-1 —CO₂R^{b} group.

Another subset of compounds that is of interest is described with respect to formula I wherein R^{a} is selected from the group consisting of: H, Phenyl, Cl and CF₃.

Another subset of compounds that is of interest is described with respect to formula I wherein ring A represents a member selected from the group consisting of Aryl, HAR, C₃₋₇cycloalkyl, C₅₋₇cycloalkyl fused to an Aryl or HAR group, Aryl and HAR fused to C₅₋₇cycloalkyl.

Another subset of compounds that is of interest is described with respect to formula I wherein ring A represents Aryl, HAR or C₃₋₇cycloalkyl.

Another subset of compounds that is of interest is described with respect to formula I wherein ring A represents a member selected from the group consisting of: phenyl, a 5-10 membered heteroaryl group selected from the group consisting of pyridyl, pyrimidyl, pyrazolyl and thienyl and a C₃₋₅cycloalkyl group.

Another subset of compounds that is of interest is described with respect to formula I wherein ring A represents a member selected from the group consisting of phenyl, pyridyl and a C₃₋₅cycloalkyl group.

Another subset of compounds that is of interest is described with respect to formula I wherein ring A represents a phenyl or cyclopropyl ring.

Another subset of compounds that is of interest is described with respect to formula I wherein ring A represents cyclopropyl.

Another subset of compounds that is of interest is described with respect to formula I wherein ring B represents a 7-8 membered bicyclic heterocyclic group containing one nitrogen atom.

Another subset of compounds that is of interest is described with respect to formula I wherein ring B represents a member selected from the group consisting of:

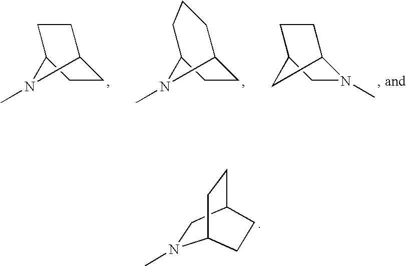

Another subset of compounds that is of interest is described with respect to formula I wherein R¹ is selected from the group consisting of H and CH₃.

Another subset of compounds that is of interest is described with respect to formula I wherein R² is selected from the group consisting of: Aryl(R^{x})ₚ in which the Aryl portion represents phenyl, p is an integer of 1-5, and each R^{x} is hydrogen, or 1-2 R^{x} groups represent halo, C₁₋₃alkyl, haloC₁₋₃alkyl, OC₁₋₃alkyl or haloOC₁₋₃alkyl, and any remaining R^{x} groups represent hydrogen.

Another subset of compounds that is of interest is described with respect to formula I wherein R² represents Aryl, which is phenyl, and all R^{x} groups represent hydrogen.

Another subset of compounds that is of interest is described with respect to formula I wherein R² represents HAR(R^{x})_{q}, q is an integer of from 1-4, HAR represents a 5-6 membered heteroaryl ring with one nitrogen atom, 0-1 oxygen or sulfur atom, and 0-2 additional nitrogen atoms, and each R^{x} group represents hydrogen, or 1-2 R^{x} groups represent a member selected from the group consisting of: methyl, ethyl, cyclopropyl, methylamino, dimethylamino, methoxy, ethoxy,

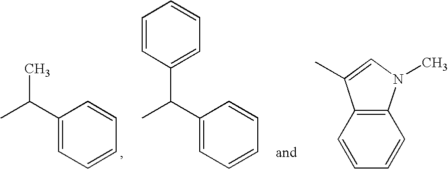

and any remaining R^{x} groups represent hydrogen.

Another subset of compounds that is of interest is described with respect to formula I wherein:

R² represents HAR(R^{x})_{q};

q represents an integer from 1-4;

HAR is selected from the group consisting of pyridyl and oxadiazolyl, and

the R^{x} groups represent hydrogen, or 1-2 R^{x} groups represent a member selected from the group consisting of methyl, ethyl, cyclopropyl, methylamino, dimethylamino, methoxy, ethoxy,

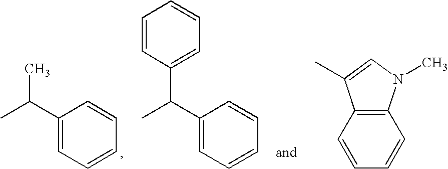

and any remaining R^{x} groups represent hydrogen.

Another subset of compounds that is of interest is described with respect to formula I wherein each R^{x} is hydrogen, or 1 R^{x} group is selected from the group consisting of C₁₋₃alkyl(R^{b})₃; OC₁₋₃alkyl(R^{b})₃; N(C₁₋₃alkyl(R^{b})₃)₂; Aryl and HAR each optionally substituted with 1-3 halo, C₁₋₃alkyl, haloC₁₋₃alkyl, OC₁₋₃alkyl or OhaloC₁₋₃alkyl groups; in which each R^{b} represents hydrogen, or 1-2 R^{b} groups represents methyl, ethyl or phenyl, 0-1 represent methoxy or ethoxy, and any remaining R^{b} groups represent hydrogen.

A subset of compounds that is of particular interest is described with respect to formula I:

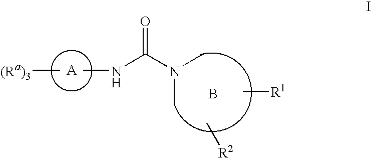

or a pharmaceutically acceptable salt or solvate thereof wherein:

ring A represents Aryl, HAR or C₃₋₇cycloalkyl;

ring B represents a 7-8 membered bicyclic heterocyclic group containing one nitrogen atom;

R^{a} is selected from the group consisting of: H, Cl, F, CH₃, CF₃, OCF₃ and Aryl that is optionally substituted with 1-3 halo, C₁₋₃alkyl, haloC₁₋₃alkyl, OC₁₋₃alkyl or OhaloC₁₋₃alkyl groups, and 0-1 —CO₂R^{b} group;

R¹ is selected from the group consisting of H and CH₃

R² is selected from the group consisting of:

Aryl(R^{x})ₚ in which the Aryl portion represents phenyl, p is an integer of 1-5, and each R^{x} is hydrogen, or 1-2 R^{x} groups represent halo, C₁₋₃alkyl, haloC₁₋₃alkyl, OC₁₋₃alkyl and haloOC₁₋₃alkyl and any remaining R^{x} groups represent hydrogen, and

HAR(R^{x})_{q}; wherein q is an integer of from 1-4, HAR represents a 5-6 membered heteroaryl ring with one nitrogen atom, 0-1 oxygen or sulfur atom, and 0-2 additional nitrogen atoms, and each R^{x} group represents hydrogen, or 1-2 R^{x} groups represent a member selected from the group consisting of: methyl, ethyl, cyclopropyl, methylamino, dimethylamino, methoxy, ethoxy,

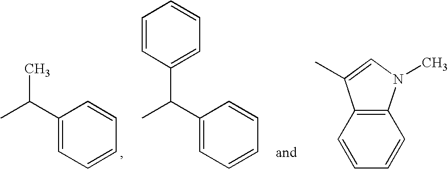

and any remaining R^{x} groups represent hydrogen.

Examples of particular compounds that fall within the invention described herein include those shown in Table 1:

TABLE 1

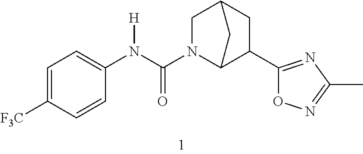

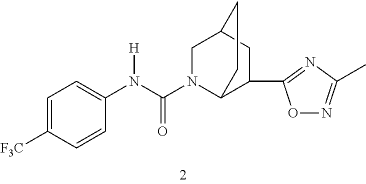

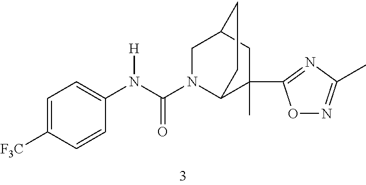

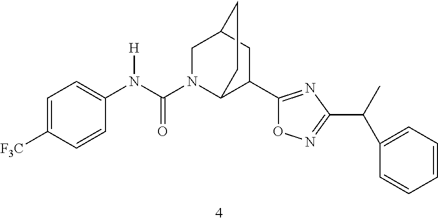

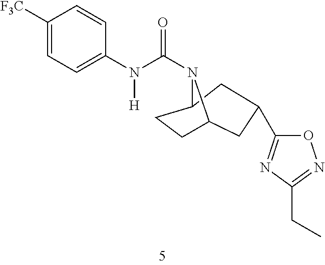

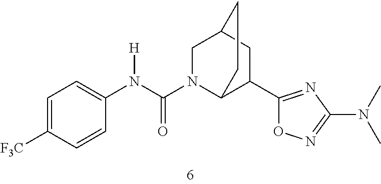

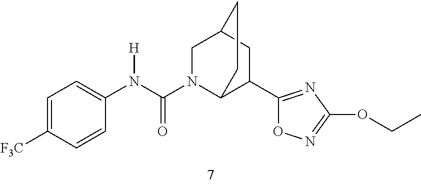

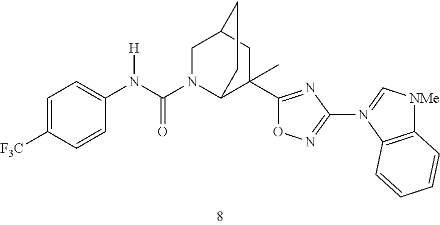

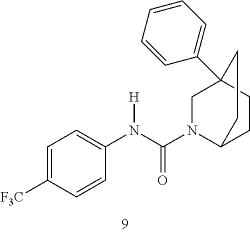

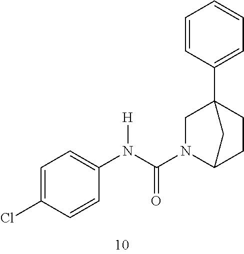

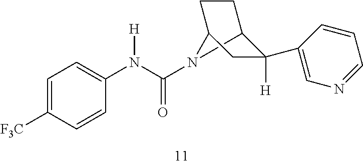

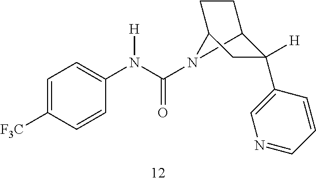

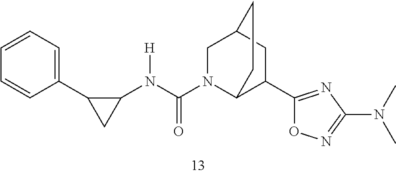

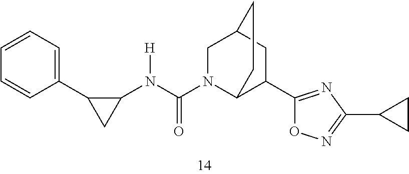

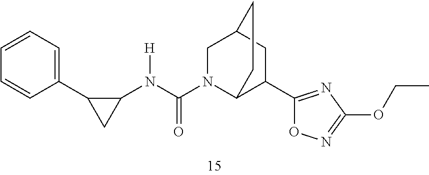

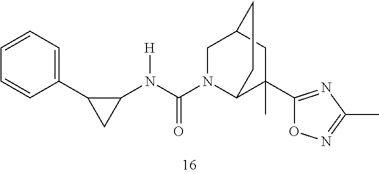

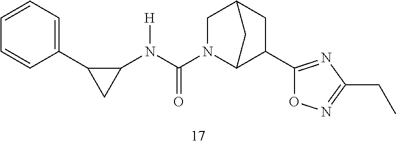

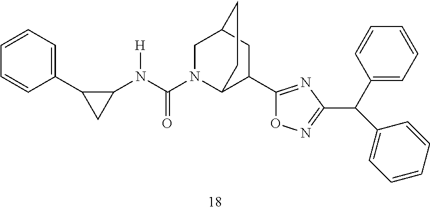

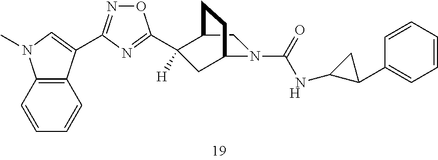

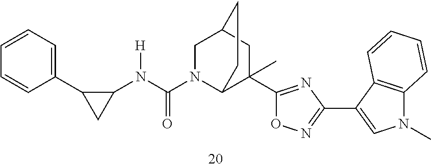

as well as the pharmaceutically acceptable salts and solvates thereof.

Yet another aspect of the invention that is of interest relates to a pharmaceutical composition comprised of a compound of formula I or a pharmaceutically acceptable salt or solvate thereof in combination with a pharmaceutically acceptable carrier.

Yet another aspect of the invention that is of interest relates to a method of treating diabetes in a mammalian patient in need of such treatment comprising administering to the patient a compound of formula I or a pharmaceutically acceptable salt or solvate thereof in an amount that is effective for treating diabetes.

Yet another aspect of the invention that is of interest relates to a method of treating pain in a mammalian patient in need of such treatment comprising administering to the patient a compound of formula I or a pharmaceutically acceptable salt or solvate thereof in an amount that is effective for treating pain.

Yet another aspect of the invention that is of interest relates to a method of treating atherosclerosis in a mammalian patient in need of such treatment comprising administering to the patient a compound of formula I or a pharmaceutically acceptable salt or solvate thereof in an amount that is effective for treating atherosclerosis.

Yet another aspect of the invention that is of interest relates to a method of treating hypertension in a mammalian patient in need of such treatment comprising administering to the patient a compound of formula I or a pharmaceutically acceptable salt or solvate thereof in an amount that is effective for treating hypertension.

Many of the compounds of formula I contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol faun are keto-enol tautomers. Or for example, a 2-hydroxyquinoline can reside in the tautomeric 2-quinolone form. The individual tautomers as well as mixtures thereof are included.

### Administration and Dose Ranges

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus and/or hyperglycemia or hypertriglyceridemia or other diseases for which compounds of Formula I are indicated, generally satisfactory results are obtained when the compounds of the present invention are administered at a dosage of from about 0.05 milligrams to about 100 milligrams per kilogram of animal body weight, preferably given as a daily dose, or in sustained release form. For most large mammals, including humans (e.g. a 70 kg adult), the total dosage administered is from about 0.1 milligrams to about 1000 milligrams, is likely to be from about 0.5 milligrams to about 350 milligrams, and is often from about 1 milligram to about 50 milligrams. For a particularly potent compound, the dosage for an adult human may be as low as 0.1 mg. Examples of dosages for a 70 kg adult human are 0.1 mg, 0.5 mg, 1 mg, 2 mg, 5 mg, 10 mg, 25 mg, 50 mg, 100 mg, 150 mg, 200 mg, 250 mg, 350 mg, and 500 mg per day. The dosage regimen may be adjusted within the above ranges or even outside of these ranges to provide the optimal therapeutic response.

Oral administration will usually be carried out using tablets. Examples of doses in tablets which may be administered include about 0.1 mg, 0.5 mg, 1 mg, 2 mg, 5 mg, 10 mg, 25 mg, 50 mg, 100 mg, 150 mg, 200 mg, 250 mg, 350 mg, and 500 mg. Other oral forms (e.g. capsules or suspensions) can be administered in doses having similar sizes.

Dosing can be carried out on a daily basis, such as once, twice or three times daily, or less often, such as every other day, every third day, once weekly or even once monthly.

### Combination Therapy

Compounds of Formula I may be used in combination with other drugs that may also be useful in the treatment or amelioration of one or more of the diseases or conditions for which compounds of Formula I are useful. Such other drugs may be administered, by a route and in an amount commonly used therefore, contemporaneously (such as via co-administration) or sequentially with a compound of Formula I. When a compound of Formula I is used contemporaneously with one or more other drugs, a pharmaceutical composition in unit dosage form containing such other drugs and the compound of Formula I is preferred. However, the combination therapy also includes therapies in which the compound of Formula I and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of Formula I, and either administered separately or in the same pharmaceutical composition, include, but are not limited to:

(a) PPAR gamma agonists and partial agonists, such as the glitazones (e.g. troglitazone, pioglitazone, englitazone, MCC-555, rosiglitazone, balaglitazone, netoglitazone, and the like), and PPAR gamma agonists and partial agonists that do not have a glitazone structure (e.g. K-111, INT-131, MBX-102 [metaglidisen], MBX-2044, FK614 including SPPARγM GSK-376501 and the like);

(b) biguanides such as metformin and phenformin;

(c) protein tyrosine phosphatase-1B (PTP-1B) inhibitors,

(d) dipeptidyl peptidase IV (DPP-4) inhibitors, including sitagliptin, vildagliptin, saxagliptin, as well as those disclosed in the following published patents and applications: U.S. Pat. No. 6,699,871; U.S. Pat. No. 7,101,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-4 inhibitor compounds include isoleucine thiazolidide (P32/98); NVP-DPP-728; vildagliptin (LAF 237); P93/01; and saxagliptin (BMS 477118).

Additional specific DPP-IV inhibitors that are of interest herein include:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5S)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl)tetrahydro-2H-pyran-3-amine;
(2R,3S,55)-2-(2,5-difluorophenyl)tetrahydro)-5-(4,6-dihydropyrrolo[3,4-c]pyrazol-5(1H)-yl)tetrahydro-2H-pyran-3-amine;
2R,3S,5R)-2-(2,4,5-trifluorophenyl)tetrahydro)-5-(4,6-dihydropyrrolo[3,4-c]pyrazol-5(1H)-yl)tetrahydro-2H-pyran-3-amine; and
(2R,3S,5S)-2-(2,4,5-trifluorophenyl)tetrahydro)-5-(4,6-dihydropyrrolo[3,4-c]pyrazol-5(1H)-yl)tetrahydro-2H-pyran-3-amine;

(e) insulin or insulin mimetics, including rapid acting insulin, regular insulin, long acting insulin, complexed forms of insulin and the like, administered by any conventional route, such as subcutaneous, intradermal or intramuscular injection, oral, transdermal, intranasal, intrapulmonary, and the like;

(f) insulin secretagogues, such as sulfonylureas (e.g. tolbutamide, glimepiride, glicazinde, and glipizide) and meglitinides (eg. repaglinide and nateglinide);

(g) α-glucosidase inhibitors (such as acarbose and miglitol);

(h) agents which improve a patient's lipid profile, such as (i) HMG-CoA reductase inhibitors (lovastatin, simvastatin, rosuvastatin, pravastatin, fluvastatin, atorvastatin, rivastatin, itavastatin, ZD-4522 and other statins), (ii) bile acid sequestrants (cholestyramine, colestipol, and dialkylaminoalkyl derivatives of a cross-linked dextran), (iii) nicotinyl alcohol, nicotinic acid (niacin) or a salt thereof, (iv) niacin receptor agonists, (v) PPARα agonists such as fenofibric acid derivatives (gemfibrozil, clofibrate, fenofibrate and bezafibrate), (vi) cholesterol absorption inhibitors, such as for example ezetimibe, (vii) acyl CoA:cholesterol acyltransferase (ACAT) inhibitors, such as avasimibe, (viii) CETP inhibitors, such as torcetrapib, ITT-705, and compounds disclosed in WO2005/100298, WO2006/014357, and WO2006/014413, and (ix) phenolic anti-oxidants, such as probucol;

(i) antiobesity compounds such as fenfluramine, dexfenfluramine, phentermine, sibutramine, orlistat, exentin-4, neuropeptide Y5 inhibitors, MC4R agonists; cannabinoid receptor 1 (CB-1) antagonists/inverse agonists, such as rimonabant and taranabant, and β₃ adrenergic receptor agonists;

(j) ileal bile acid transporter inhibitors;

(k) agents intended for use in inflammatory conditions such as aspirin, non-steroidal anti-inflammatory drugs as further described below, glucocorticoids, azulfidine, and cyclooxygenase 2 selective inhibitors;

(l) glucagon receptor antagonists;

(m) GLP-1;

(n) GIP-1; and

(o) GLP-1 analogs, such as exentin-4, including exenatide;

(p) GPR 119 agonists;

(q) 11-B HSD 1 inhibitors;

(r) glucokinase activators;

(s) SGLT, particularly SGLT2 inhibitors;

(t) PPARδ agonists such as those disclosed in WO 97/28149;

(u) prandial glucose releasing agents such as repaglinide and nateglinide,

(v) antihypertensives, such as diuretics, e.g., hydrochlorothiazide, furosemide and the like; beta adrenergic blocking drugs, such as propranolol, metaprolol and the like; ACE inhibitors, such as enalapril, lisinopril, ramipril, quinapril and the like, ARBs, such as losartan, valsartan, irbesartan, candesartan and the like, and calcium channel blocking drugs, such as amlodipine, diltiazem and verapamil; and

(w) NSAIDS such as ibuprofen, naproxen, meloxicam, diclofenac, indomethacin, piroxicam, COX-2 inhibitors such as nabumetone, etodolac, rofecoxib, etoricoxib, celecoxib, and valdecoxib, and conventional non-opioid and opioid analgesics, such as aspirin, acetaminophen, codeine, meperidine, oxycodone, hydrocodone, pentazocine, morphine and the like.

The above combinations include combinations of a compound of the present invention not only with one other active compound, but also with two or more other active compounds. Non-limiting examples include combinations of compounds having Formula I with two or more active compounds selected from biguanides, sulfonylureas, HMG-CoA reductase inhibitors, other PPAR agonists, PIP-1B inhibitors, DPP-4 inhibitors, and anti-obesity compounds.

Examples of glucagon receptor antagonist compounds that are useful as described herein include: N-[4-((1S)-1-{3-(3,5-Dichlorophenyl)-5-[6-(trifluoromethoxy)-2-naphthyl]-1H-pyrazol-1-yl}ethyl)benzoyl]-β-alanine;
N-[4-((1R)-1-{3-(3,5-Dichlorophenyl)-5-[6-(trifluoromethoxy)-2-naphthyl]-1H-pyrazol-1-yl}ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-Dichlorophenyl)-5-(6-methoxy-2-naphthyl)-1H-pyrazol-1-yl]ethyl}benzoyl)-β-alanine;
3-({4-[(2S)-2-({5-chloro-[3-(trifluoromethyl)phenyl]-1H-indol-2-yl}carbonyl)heptyl]benzoyl}amino)propanoic acid and 3-({4-[(2R)-2-({5-chloro-1-[3-(trifluoromethyl)phenyl]-1H-indol-2-yl}carbonyl)heptyl]benzoyl}amino)propanoic acid;
3-[(3-Bromo-4-{(2S)-2-[(5-chloro-1-methyl-1H-indol-2-yl)carbonyl]heptyl}benzoyl)amino]propanoic acid and 3-[(3-Bromo-4-{(2R)-2-[(5-chloro-1-methyl-1H-indol-2-yl)carbonyl]heptyl}benzoyl)amino]propanoic acid;
3-{[4-((2R)-2-{[1-(4-tert-butylbenzyl)-5-chloro-1H-indol-2-yl]carbonyl}pentyl)benzoyl]amino}propanoic acid and 3-{[4-((2S)-2-{[1-(4-tert-butylbenzyl)-5-chloro-1H-indol-2-yl]carbonyl}pentyl)benzoyl]amino}propanoic acid;
3-{[4-((2R)-2-{[5-Chloro-1-(3,5-dichlorophenyl)-1H-indol-2-yl]carbonyl}pentyl)benzoyl]amino}propanoic acid and 3-{[4-(2S)-2-{[5-Chloro-1-(3,5-dichlorophenyl)-1H-indol-2-yl]carbonyl}pentyl)benzoyl]amino}propanoic acid;
N-(4-{(1S)-1-[3-[2-Ethoxy-5-(trifluoromethyl)phenyl]-5-(6-methoxy-2-naphthyl)-1H-pyrazol-1-yl]ethyl}benzoyl)-β-alanine;
N-(4-{(1S)-1-[3-[2-methoxy-5-(trifluoromethyl)phenyl]-5-(6-methoxy-2-naphthyl)-1H-pyrazol-1-yl]ethyl}benzoyl)-β-alanine;
N-[4-((1S)-1-{5-(6-methoxy-2-naphthyl)-3-[2-methoxy-5-(trifluoromethyl)phenyl]-1H-pyrazol-1-yl}pentyl)benzoyl]-β-alanine;
N-[4-((1S)-1-{5-(6-chloro-2-naphthyl)-3-[2-methoxy-5-(trifluoromethyl)phenyl]-1H-pyrazol-1-yl}pentyl)benzoyl]-β-alanine;
N-(4-{(1S)-1-[(R)-(4-chlorophenyl)(7-fluoro-5-methyl-1H-indol-3-yl)methyl]butyl}benzoyl)-β-alanine;
N-(4-{1-[(4-chlorophenyl)(5,7-dichloro-1H-indol-3-yl)methyl]butyl}benzoyl)-β-alanine;
N-(4-{1-[(3-chloro-4-methoxyphenyl)(5,7-dichloro-1H-indol-3-yl)methyl]pentyl}benzoyl)-β-alanine; and
N-(4-{1-[(5,7-dichloro-1H-indol-3-yl)(3,5-dichloro-4-methoxyphenyl)methyl]pentyl}benzoyl)-β-alanine.

Examples of GPR-119 agonists that are of interest as described herein include:
4-Methyl-6-[1′-(5-methylpyrazin-2-yl)-4,4′-bipiperidin-1-yl]pyrimidine-2-carbonitrile;
1-(5-chloropyrazin-2-yl)-1′-[5-(methylsulfonyl)pyridin-2-yl]-4,4′-bipiperidine;
2-chloro-4-(1′-pyrimidin-2-yl-4,4′-bipiperidin-1-yl)benzonitrile and 1-(5-chloro-2-methylpyrimidin-4-yl)-1′-(5-chloropyrimidin-2-yl)-4,4′-bipiperidine.

Also claimed is the use of additional PPAR alpha, gamma or delta selective agonists, PPAR alpha/gamma, gamma/delta, alpha/delta dual agonists, or PPAR alpha/gamma/delta pan agonists. These agents are useful for the treatment of diabetes, dyslipidemia and weight loss. Examples of such agents include, but are not limited to the following: netoglitazone, pioglitazone, rosiglitazone, troglitazone, balaglitazone, CS204, AZD6610, ZYH1, GFT505, LY-465608, DRF-2519, DRF-11605, DRF-2725, GW-626019, GW-625019, CS038, ONO-5129, aleglitazar, muraglitazar, soldeglitazar, teseglitazar, naveglitazar, farglitazar, KRP-297, AVE0897, AVE 0847, LBM642, PPM263, PPM202, PPM201, PPM204, PLX-204, GW-677954, NN0606, AVE8134, NS-220, SAR 35034, KD3010, GW-501516, FK614, K-111, metaglidasen, MBX-2044, INT-131, KD3010, KR-62980, SVT002149, AVE8134, AVE5378, AVE0897, SAR35034, AVE5376, MBX2130, PAT-5A, GW-501516, GW-1262570, GW677954, GW590735, R-483, and BAY-54-9801.

Examples of SPPARMs that are of interest as described herein include:
(2S)-2-({6-chloro-3-[6-(4-chlorophenoxy)-2-propylpyridin-3-yl]-1,2-benzisoxazol-5-yl}oxy)propanoic acid;
(1S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid.

Examples of 11B-HSD 1 inhibiting compounds that are of interest as described herein include:
3-[1-(4-chlorophenyl)-trans-3-fluorocyclobutyl]-4,5-dicyclopropyl-r-4H-1,2,4-triazole; 3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(Ethylsulfonyl)propyl]bicyclo[2.2.2]oct-1-yl}-4-methyl-5-[2-(trifluoromethyl)phenyl]-4H-1,2,4-triazole;
4-Methyl-3-{4-[4-(methylsulfonyl)phenyl]bicyclo[2.2.2]oct-1-yl}-5-[2-(trifluoromethyl)phenyl]-4H-1,2,4-triazole;
3-(4-{4-Methyl-5-[2-(trifluoromethyl)phenyl]-4H-1,2,4-triazol-3-yl}bicycle[2.2.2]oct-1-yl)-5-(3,3,3-trifluoropropyl)-1,2,4-oxadiazole;
3-(4-{4-Methyl-5-[2-(trifluoromethyl)phenyl]-4H-1,2,4-triazol-3-yl}bicycle[2.2.2]oct-1-yl)-5-(3,3,3-trifluoroethyl)-1,2,4-oxadiazole;
5-(3,3-Difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-Fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-Difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-Difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-Difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole.

Examples of glucokinase activating drugs that are of interest for use as described herein include:
6-(1-acetylpyrrolidin-2-yl)-5-(6-methoxymethylpyridin-3-yl)oxy)-2-pyridin-2-yl-1H-benzimidazole
6-(1-acetylpyrrolidin-2-yl)-5-(6-methylpyridin-3-yl)oxy)-2-pyridin-2-yl-1H-benzimidazole,
6-(1-acetylpyrrolidin-2-yl)-5-((6-pyrazin-2-ylpyridin-3-yl)oxy)-2-pyridin-2-yl-1H-benzimidazole,
6-(1-acetyl-3-fluoropyrrolidin-2-yl)-5-((2′-fluorobiphenyl-4-yl)oxy)-2-pyridin-2-yl-1H-benzimidazole,
3-(6-ethanesulfonyl-pyridin-3-yloxy)-5-(2-hydroxy-1-methyl-ethoxy)-N-(1-methyl-1H-pyrazol-3-yl)benzamide;
3-(6-ethanesulfonyl-pyridin-3-yloxy)-5-isopropoxy-N-(1-methyl-1H-pyrazol-3-yl)benzamide;
5-(2-fluoro-1-fluoromethyl-ethoxy)-3-(6-methanesulfonyl-pyridin-3-yloxy)-N-(1-methyl-1H-pyrazol-3-yl)benzamide;
3-(6-ethanesulfonyl-pyridin-3-yloxy)-5-(2-hydroxy-1-methyl-ethoxy)-N-(isoxazol-3-yl)benzamide;
1-[(5-{[6-(5-methyl-1,2,4-oxadiazol-3-yl)-3-pyridinyl]oxy}-2-(2-pyridinyl)-1H-benzimidazol-6-yl)methyl]-2-pyrrolidinone,
N-({5-[4-ethylsulfonyl)phenoxy]-2-(2-pyridinyl)-1H-benzimidazol-6-yl}methyl)-N-methylacetamide,
3-{[5-[4-(5-methyl-1,2,4-oxadiazol-3-yl)phenoxy]-2-(2-pyridinyl)-1H-benzimidazol-6-yl]methyl}-1,3-oxazolidine-2,4-dione,
5-[4-(ethylsulfonyl)phenoxy]-6-((2-methyl-2H-tetrazol-5-yl)methyl)-2-(2-pyridinyl)-1H-benzimidazole,
3-({4-[2-(dimethylamino)ethoxy]phenyl}thio)-N-(3-methyl-1,2,4-thiadiazol-5-yl)-6-[(4-methyl-4H-1,2,4-triazol-3-yl)thio]pyridine-2-carboxamide;
3-({4-[(1-methylazetidin-3-yl)oxy]phenyl}thio)-N-(3-methyl-1,2,4-thiadiazol-5-yl)-6-[(4-methyl-4H-1,2,4-triazol-3-yl)thio]pyridine-2-carboxamide;
3-{[4-(2-methoxyethoxy)phenyl]thio}-N-(3-methyl-1,2,4-thiadiazol-5-yl)-6-[(4-methyl-4H-1,2,4-triazol-3-yl)thio]pyridine-2-carboxamide; and
3-[(4-acetylphenyl)thio]-N-(3-methyl-1,2,4-thiadiazol-5-yl)-6-[(4-methyl-4H-1,2,4-triazol-3-yl)thio]pyridine-2-carboxamide.

Compounds of the present invention (i.e. compounds having Formula I) can be used to treat one or more diseases or conditions selected from hypercholesterolemia, atherosclerosis, low HDL levels, high LDL levels, hyperlipidemia, hypertriglyceridemia, and dyslipidemia by administering a therapeutically effective amount of a compound of claim 1 in combination with an HMG-CoA reductase inhibitor to a patient in need of such treatment. Statins are the preferred HMG-CoA reductase inhibitors for use in this combination therapy. Preferred statins include lovastatin, simvastatin, pravastatin, fluvastatin, atorvastatin, itavastatin, ZD-4522, rivastatin, and rosuvastatin. This combination treatment may be particularly desirable for treating or reducing the risk of developing atherosclerosis. Such a combination can optionally have a third pharmaceutically active ingredient, such as a CETP inhibitor (e.g. torcetrapib), niacin, or a cholesterol absorption inhibitor (e.g. ezetimibe).

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications as outlined above. For example, a diabetic patient receiving treatment as described herein may also be taking insulin or an oral antidiabetic medication. One example of an oral antidiabetic medication useful herein is metformin.

For hypertensive patients, the compounds used in the present invention can be administered with conventional antihypertensive medications as outlined above. For example, a patient with high blood pressure receiving treatment as described herein may also be taking ARBS or an ACE inhibitor. One example of an oral antihypertensive medication useful herein is losartin.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic or has a basic group in the structure, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, muck, nitric, pamoic, pantothenic, phosphoric, succinic, sulfuric, tartaric, p-toluenesulfonic acid, and the like. Preferred acids include citric, hydrobromic, hydrochloric, maleic, phosphoric, sulfuric, tartaric, toluenesulfonic (tosylate), methanesulfonic (mesylate) and benzenesulfonic (besylate) acid salts, most preferably the benzenesulfonic, toluenesulfonic and methanesulfonic acid salts. In some instances the compounds of the invention may be present in zwitterionic forms.

It will be understood that, as used herein, references to the compounds of Formula I are meant to also include the pharmaceutically acceptable salts.

### Metabolites—Prodrugs

Metabolites of the claimed compounds which themselves fall within the scope of the claimed invention are also compounds of the current invention. Prodrugs, which are metabolically or physically labile compounds that are converted to the claimed active pharmaceutical ingredient (API) as they are being administered to a patient or after they have been administered to a patient, also may be considered compounds of this invention.

### Pharmaceutical Compositions

The pharmaceutical compositions described herein are generally comprised of a compound of formula I or a pharmaceutically acceptable salt or solvate thereof, in combination with a pharmaceutically acceptable carrier.

The compounds used in the present invention can be administered via any conventional route of administration. The preferred route of administration is oral. Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

One embodiment of the invention that is of interest is a tablet or capsule that is comprised of a compound of formula I or a pharmaceutically acceptable salt or solvate thereof in an amount ranging from about 0.1 mg to about 1000 mg, in combination with a pharmaceutically acceptable carrier.

In another embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 0.1 to about 1000 mg) and the second therapeutic agent (about 0.1 to about 500 mg) are combined with the pharmaceutically acceptable carrier, providing a tablet or capsule for oral use. Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents,e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs are also included.

More particularly, a pharmaceutical composition that is of interest is a sustained release tablet that is comprised of a compound of formula I or a pharmaceutically acceptable salt or solvate thereof in combination with a pharmaceutically acceptable carrier.

Another aspect of the invention relates to the use of a compound of formula I or a pharmaceutically acceptable salt or solvate thereof in the manufacture of a medicament. This medicament has the uses described herein.

More particularly, another aspect of the invention relates to the use of a compound of formula I or a pharmaceutically acceptable salt or solvate thereof, and an HMG Co-A reductase inhibitor, such as simvastatin, in the manufacture of the medicament. This medicament has the uses described herein.

### Utilities

The compounds defined above may be used in any of the following methods to treat or control diseases, as well as methods to treat other diseases not listed below, in a mammalian patient, especially a human, by administering to the patient a therapeutically effective amount for the specific disease (or diseases) of a compound of Formula I:

(1) non-insulin dependent diabetes mellitus (type 2 diabetes);

(2) pre-diabetes (insulin resistance);

(3) hyperglycemia;

(4) metabolic syndrome;

(5) obesity;

(6) atherosclerosis;

(7) hypertension;

(8) one or more lipid disorders, including mixed or diabetic dyslipidemia, hyperlipidemia, and hypercholesterolemia;

(9) glaucoma, age related macular degeneration and the like;

(10) organ protection, such as protection from reperfusion injury; and

(11) kidney malfunction, such as proteinuria, and in particular, albuminuria, and subsequent edema resulting therefrom, macrophage infiltration, and the like.

The compounds may also be used in a method for reducing the risks of adverse sequelae associated with metabolic syndrome in a human or other mammalian patient in need of such treatment which comprises administering to the patient a therapeutically effective amount of a compound of Formula I, or a pharmaceutically acceptable salt thereof.

The compounds may also be used in a method for treating atherosclerosis, for reducing the risk of developing atherosclerosis, for delaying the onset of atherosclerosis, and/or reducing the risk of sequelae of atherosclerosis in a human or other mammalian patient in need of such treatment or at risk of developing atherosclerosis or sequelae of atherosclerosis, which comprises administering to the patient a therapeutically effective amount of a compound of Formula I. Sequelae of atherosclerosis include for example angina, claudication, heart attack, stroke, etc.

The compounds are especially useful in the treatment of the following diseases, by administering a therapeutically effective amount (for the specific disease) of the compound, or a pharmaceutically acceptable salt thereof, to a patient in need of treatment:

(1) type 2 diabetes, and especially insulin resistance resulting from type 2 diabetes;

(2) hypertension;

(3) atherosclerosis; and

(4) metabolic syndrome.

Another aspect of the invention that is of interest relates to a method of treating atherosclerosis in a human patient in need of such treatment comprising administering to the patient a compound of formula I or a pharmaceutically acceptable salt or solvate thereof in an amount that is effective for treating atherosclerosis.

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of interest relates to a method of treating high blood pressure in a human patient in need of such treatment comprising administering to the patient a compound of formula I or a pharmaceutically acceptable salt or solvate thereof in an amount that is effective for treating hypertension.

Another aspect of the invention that is of interest relates to a method of treating inflammatory pain or CNS-mediated pain in a human patient in need of such treatment comprising administering to the patient a compound of formula I or a pharmaceutically acceptable salt or solvate thereof in an amount that is effective for treating pain.

Another aspect of the invention that is of interest relates to a method of treating disorders of the eye in a human patient in need of such treatment comprising administering to the patient a compound of formula I or a pharmaceutically acceptable salt or solvate thereof in an amount that is effective for alleviating eye disorders.

Another aspect of the invention that is of interest relates to a method of treating cardiac hypertrophy and renal failure in a human patient in need of such treatment comprising administering to the patient a compound of formula I or a pharmaceutically acceptable salt or solvate thereof in an amount that is effective for anti-inflammatory end organ protection.

Another aspect of the invention that is of particular interest relates to a method of treating or preventing atherosclerosis, diabetes, hypertension, metabolic syndrome or a related condition in a human patient in need of such treatment, comprising administering to the patient a compound of formula I or a pharmaceutically acceptable salt or solvate thereof administered in an amount that is effective to treat or prevent atherosclerosis, diabetes, hypertension, metabolic syndrome or a related condition.

Compounds of the present invention are inhibitors of the enzyme, soluble epoxide hydrolase (sEH). The compounds of this invention are useful in treating or controlling diseases, disorders or conditions which are mediated by sEH and EETs (Larsen, Campbell and Gutterman TRENDS in Pharmacol. Sci. 2007, 28(1), 32). One aspect of the present invention provides a method for the treatment and control of diseases that can be mediated by administration of an sEH inhibitor, such as type 2 diabetes or hypertension. Compounds of the present invention may be useful in treating or controlling many sEH mediated diseases and conditions, including, but not limited to, (1) diabetes mellitus, and especially non-insulin dependent type 2 diabetes mellitus (NIDDM), (2) hyperglycemia, (3) low glucose tolerance, (4) pre-diabetes or insulin resistance, (5) obesity, (6) hypertension, (7) dyslipidemia, (8) hyperlipidemia, (9) hypercholesterolemia, (10) atherosclerosis and its sequelae, (11) kidney failure, (12) cardiac hypertrophy, (13) pancreatitis, (14) vascular restenosis, (15) inflammatory pain, (16) CNS-mediated pain, (17) glaucoma, (18) macular degeneration, (19) retinopathy, (20) thrombosis, (21) metabolic syndrome, and (22) Raynaud's syndrome.

Another aspect of the invention provides a method of treating inflammatory conditions, including adult respiratory distress syndrome (ARDS), ischemia/reperfusion injury and related diseases.

The present compounds can be used to lower glucose and insulin in non-diabetic patients who have impaired glucose tolerance and/or are in a pre-diabetic condition by the administration to a patient in need of treatment a therapeutically effective amount of a compound having Formula I, or pharmaceutically acceptable salt thereof.

The present compounds can be used to treat obesity in a patient in need of such treatment by administering to the patient a therapeutically effective amount of a compound of Formula I, or pharmaceutically acceptable salt thereof.

The present compounds can be used to treat or reduce the risk of developing atherosclerosis in a patient in need of such treatment by administering to the patient a therapeutically effective amount of a compound of Formula I, or a pharmaceutically acceptable salt thereof.

The present compounds can be used to treat or reduce hyperglycemia in a diabetic patient in need of such treatment by administering to the patient a therapeutically effective amount of a compound of Formula I, or a pharmaceutically acceptable salt thereof.

The present compounds can be used to treat or reduce blood pressure and provide kidney end organ protection in a hypertensive patient in need of such treatment by administering to the patient a therapeutically effective amount of a compound of Formula I, or a pharmaceutically acceptable salt thereof.

One aspect of the invention provides a method for the treatment and control of mixed or diabetic dyslipidemia, and/or atherosclerosis, which comprises administering to a patient in need of such treatment a therapeutically effective amount of a compound having formula I. The compound may be used alone or advantageously may be administered with a cholesterol biosynthesis inhibitor, particularly an HMG-CoA reductase inhibitor such as lovastatin, simvastatin, rosuvastatin, pravastatin, fluvastatin, atorvastatin, rivastatin, itavastatin, or ZD-4522. The compound may also be used advantageously in combination with other lipid lowering drugs such as cholesterol absorption inhibitors (for example stanol esters, sterol glycosides such as tiqueside, and azetidinones such as ezetimibe), ACAT inhibitors (such as avasimibe), CETP inhibitors (such as torcetrapib), niacin, niacin receptor agonists, bile acid sequestrants, microsomal triglyceride transport inhibitors, and bile acid reuptake inhibitors. These combination treatments may also be effective for the treatment or control of one or more related conditions selected from the group consisting of hypercholesterolemia, atherosclerosis, hyperlipidemia, hypertriglyceridemia, dyslipidemia, high LDL-c levels, and low HDL-c levels.

Another aspect of the invention that is of interest relates to a method of treating or controlling one or more of: mixed or diabetic dyslipidemia, hypercholesterolemia, atherosclerosis, low HDL levels, high LDL levels, hyperlipidemia, and/or hypertriglyceridemia, type 2 diabetes, hyperglycemia, insulin resistance and related conditions, hypertension, and/or kidney failure, and inflammatory pain which comprises administering to a patient in need of such treatment a therapeutically effective amount of a compound having formula I in combination with a compound selected from the group consisting of:

a DPP-4 antagonist; a glucagon receptor antagonist; a glucokinase activator; a GPR119 agonist; a GPR 40 modulator; a GPR 120 agonist; an insulin sensitizer; a sulfonylurea or other insulin secretagogue; a SPPARγM such as those disclosed in WO 2006/099077 A1; an α-glucosidase inhibitor; an SGLT inhibitor; a GLP-1, GLP-1 analogue or mimetic or a GLP-1 receptor agonist; a GIP, GIP mimetic or GIP receptor agonist; a PACAP, a PACAP mimetic or PACAP receptor agonist; an HMG Co-A reductase inhibitor; a bile acid sequestrant; (niacin) nicotinic acid or a nicotinyl alcohol; a PPAR α agonist; a PPARα/γ dual agonist; a PPAR pan agonist; inhibitors of cholesterol absorption; acyl CoA:cholesterol acyltransferase inhibitors; antioxidants; PPARδ agonists; antiobesity agents such as NPY1 or NPY5 antagonists CB1 receptor inverse agonists, ileal bile acid transporter inhibitors; aspirin, NSAIDs, glucocorticoids, azulfidine, selective COX-2 inhibitors; antihypertensive agents such as ACE inhibitors, AII receptor blockers, beta blockers and calcium channel blocking drugs; diuretics; inhibitors of 11β-HSD-1; inhibitors of CETP and inhibitors of fructose 1,6-bisphosphatase.

### Pharmaceutical Compositions

Another aspect of the present invention provides pharmaceutical compositions which comprise a compound of Formula I and a pharmaceutically acceptable carrier. The pharmaceutical compositions of the present invention comprise a compound of Formula I or a pharmaceutically acceptable salt as an active ingredient, as well as a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids. A pharmaceutical composition may also comprise a prodrug, or a pharmaceutically acceptable salt thereof, if a prodrug is administered.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy. In general, compositions suitable for oral administration are preferred.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form, in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The following are examples of pharmaceutical dosage forms containing a compound of Formula I:

| Injectable Suspension (im.) | mg/mL | |
|---|---|---|
| Compound of Formula 1 | 10.0 | |
| Methylcellulose | 5.0 | |
| Tween 80 | 0.5 | |
| Benzyl alcohol | 9.0 | |
| Benzalkonium chloride | 1.0 | |
| Water for injection | t.d. 1.0 mL | |
| Capsule | mg/capsule | |
| Compound of Formula 1 | 25.0 | |
| Lactose | 735 | |
| Mg Stearate | 1.5 | |
| Total | 600 mg | |
| Tablet | Mg/tablet | |
| Compound of Formula 1 | 25.0 | |
| Microcrystalline Cellulose | 415 | |
| Povidone | 14.0 | |
| Pregelatinized Starch | 4.35 | |
| Magnesium Stearate | 2.5 | |
| Total | 500 mg | |
| Aerosol | Per Canister | |
| Compound of Formula 1 | 250 | mg |
| Lecithin, NF Liq. Conc. | 1.2 | mg |
| Trichloromethane, NF | 4.025 | g |
| Dichlorodifluoromethane, NF | 12.15 | g |

### REPRESENTATIVE SCHEMES AND EXAMPLES

The following Schemes and Examples are provided to more fully illustrate the present invention. Representative compounds of Formula I have been prepared by the following reaction Schemes below. It is understood that other synthetic approaches to these structure classes are conceivable to one skilled in the art. Therefore these reaction Schemes, as well as the Examples, should not be construed as limiting the scope of the invention. Unless stated otherwise:

(i) all operations were carried out at room (rt) or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (1H NMR) spectrometry, and the purity was assured by at least one of the following techniques: TLC or HPLC;

(vi) 1H NMR spectra were recorded on either a Varian Unity™ or a Varian Inova™ instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vii) MS data were recorded on a Waters Micromass unit, interfaced with a Hewlett-Packard (Agilent 1100™) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid−acetonitrile, A=0.1% formic acid−water), Waters XTerra C18−3.5 um−50×3.0 mmID and diode array detection;

(viii) automated purification of compounds by preparative reverse phase RP-HPLC was performed on a Gilson system using a YMC-Pack Pro C18 column (150×20 mm i.d.) eluting at 20 mL/min with 0-50% acetonitrile in water (0.1% TFA);

(ix) column chromatography was carried out on a glass silica gel column using Kieselgel 60™, 0.063-0.200 mm (Merck), or a Biotage cartridge system;

(x) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar).

The various organic group transformations and protecting groups utilized herein can be performed by a number of procedures other than those described below. References for other synthetic procedures that can be utilized for the preparation of intermediates or compounds disclosed herein can be found in, for example, M. B. Smith, J. March Advanced Organic Chemistry, 5^{th} Edition, Wiley-Interscience (2001); R. C. Larock Comprehensive Organic Transformations, A Guide to Functional Group Preparations, 2^{nd} Edition, VCH Publishers, Inc. (1999); T. L. Gilchrist Heterocyclic Chemistry, 3^{rd} Edition, Addison Wesley Longman Ltd. (1997); J. A. Joule, K. Mills, G. F. Smith Heterocyclic Chemistry, 3^{rd} Edition, Stanley Thornes Ltd. (1998); G. R. Newkome, W. W. Paudler Contemporary Heterocyclic Chemistry, John Wiley and Sons (1982); or Wuts, P. G. M.; Greene, T. W.; Protective Groups in Organic Synthesis, 3^{rd} Edition, John Wiley and Sons, (1999), all six incorporated herein by reference in their entirety.

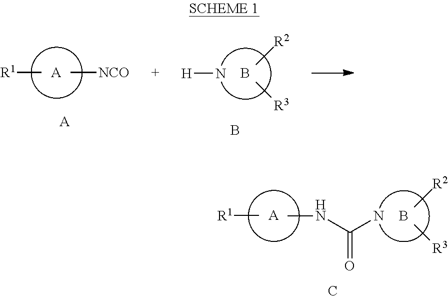

The compounds of formula I are prepared by reacting equivalent amounts of a secondary amine B with an appropriately substituted isocyanate A. This reaction is typically conducted in a suitable solvent, such as dichloromethane. The reaction is typically followed by the addition of diisopropylethyl amine (1 equivalent). The mixture is typically stirred at room temperature until completion, e.g., for about 2-14 hrs. The solvent is removed in vacuo, and the residue is purified by reverse-phase HPLC to give the desired compound of formula I.

### Specific Procedures

To a 1 dram vial containing a solution of the secondary amine shown in Table 2 below (1 equivalent) in 1 mL of dichloromethane was added the isocyanate shown in column 1 (1 equivalent), followed by the addition of diisopropylethylamine (35 μL, 26 mg). The mixture was stirred at room temperature for 14 h. After the removal of solvent in vacuo, the residue was purified by reverse-phase HPLC to give the desired product.

| Isocyanate 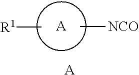 | Secondary Amine 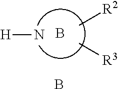 | Product 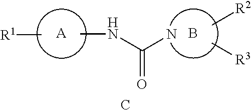 |
|---|---|---|
| 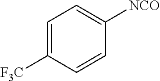 | 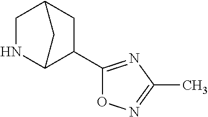 | 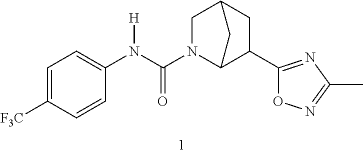 |
| 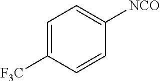 | 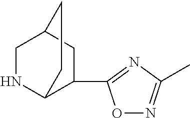 | 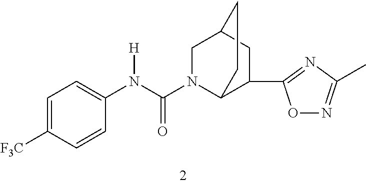 |
| 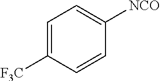 | 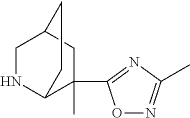 | 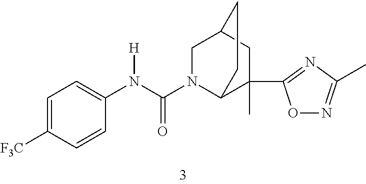 |
| 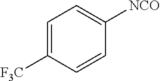 | 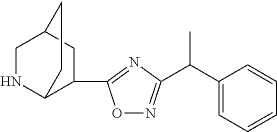 | 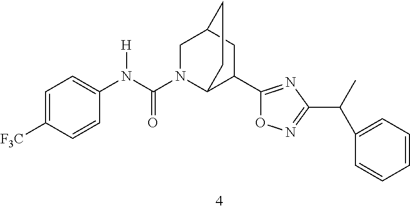 |
| 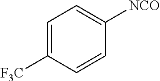 | 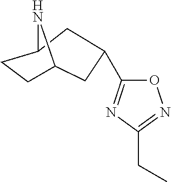 | 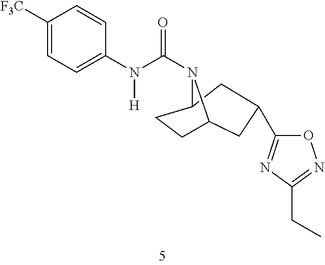 |
| 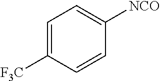 | 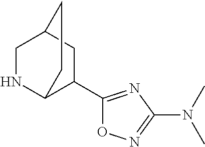 | 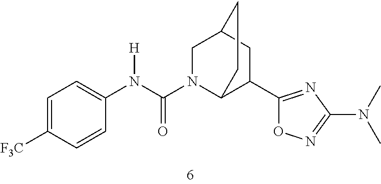 |
| 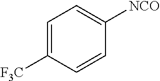 | 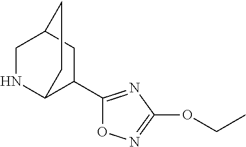 | 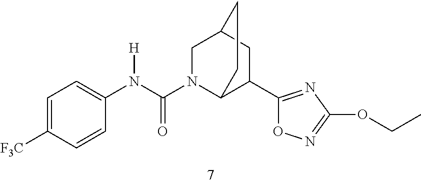 |
| 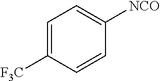 | 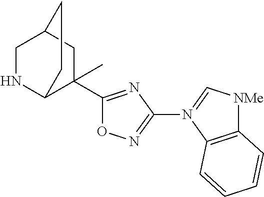 | 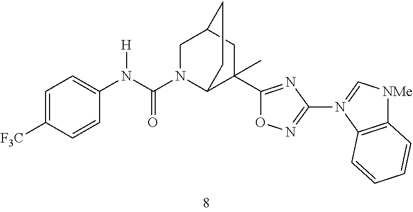 |
| 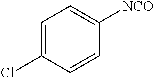 | 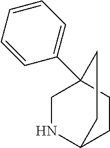 | 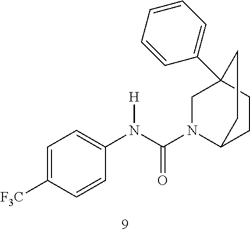 |
| 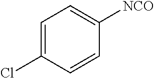 | 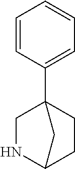 | 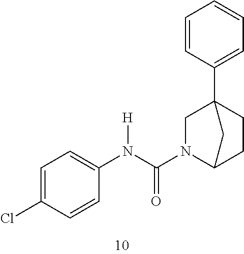 |
| 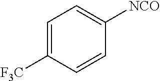 | 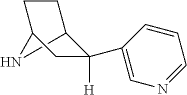 | 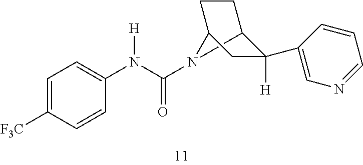 |
| 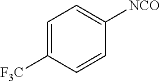 | 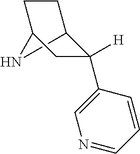 | 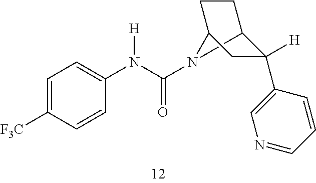 |
| 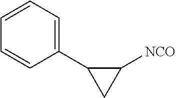 | 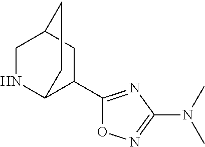 | 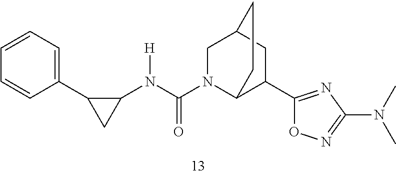 |
| 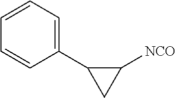 | 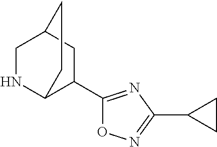 | 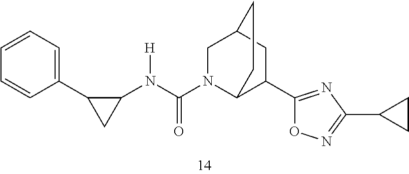 |
| 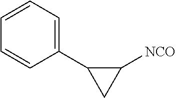 | 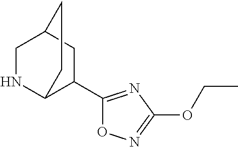 | 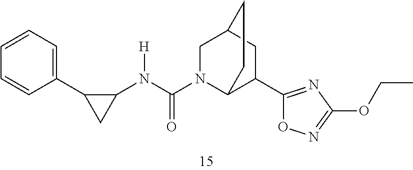 |
| 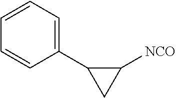 | 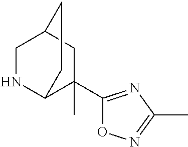 | 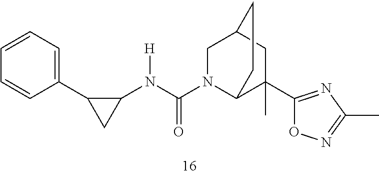 |
| 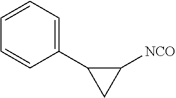 | 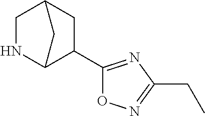 | 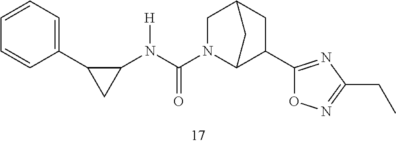 |
| 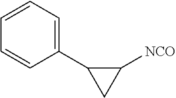 | 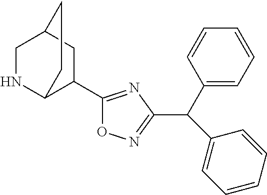 | 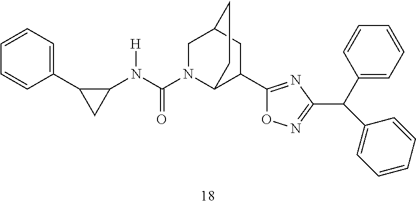 |
| 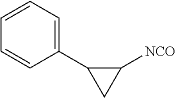 | 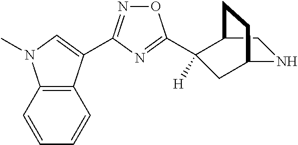 | 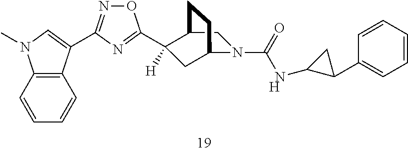 |
| 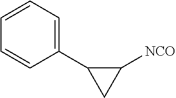 | 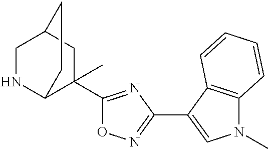 | 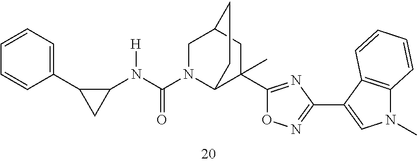 |

NMR data for the compounds is presented below.

### Example 1

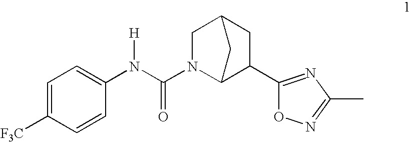

1: ¹H NMR (Acetone-d₆, 500 MHz): δ 8.19 (1H, s), 7.82 (1H, d), 7.58 (1H, d), 3.57 (2H, m), 3.33 (1H, d), 2.34 (3H, s), 2.15 (2H, m), 2.08 (2H, m), 1.86 (1H, d), 1.78 (1H, d); LCMS m/z: 367 (M⁺+1).

The starting secondary amine for this example can be prepared according to Floersheim, Philipp; et al. Isosterism and bioisosterism case studies with muscarinic agonists. Chimia, 1992, 46, 323-34.

### Example 2

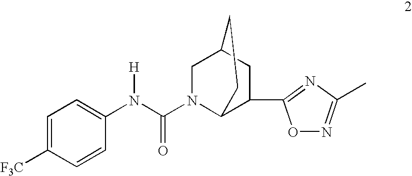

2: ¹H NMR (Acetone-d₆, 500 MHz): δ 8.08 (1H, s), 7.83 (1H, d), 7.57 (1H, d), 4.64 (1H, s), 3.67 (2H, m), 2.35 (3H, s), 2.26 (2H, m), 2.19 (1H, m), 1.82 (1H, m), 1.74 (3H, m); LCMS m/z: 381 (M⁺+1).

The starting amine for this example is prepared according to Street, Leslie J.; et al. Synthesis and biological activity of 1,2,4-oxadiazole derivatives: highly potent and efficacious agonists for cortical muscarinic receptors. Journal of Medicinal Chemistry 1990, 33, 2690-7 and Baker, Raymond; et al. Preparation of oxadiazoles useful in the treatment of senile dementia. Eur. Pat. Appl. (1987), EP 239309 A2.

### Example 3

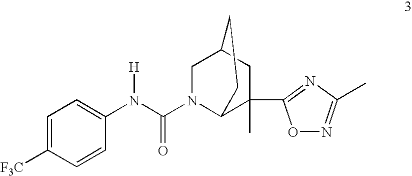

3: ¹H NMR (Acetone-d₆, 500 MHz): δ 8.10 (1H, s), 7.86 (2H, d), 7.59 (2H, d), 4.57 (1H, s), 3.67 (2H, m), 2.79 (1H, m), 2.37 (3H, s), 2.21 (1H, m), 1.82 (1H, m), 1.65 (3H, m), 1.44 (3H, s), 1.39 (1H, m); LCMS m/z: 395 (M⁺+1).

### Example 4

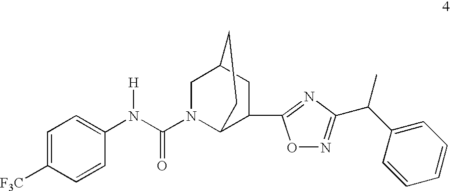

4: ¹H NMR (Acetone-d₆, 500 MHz): δ 8.09 (1H, s), 7.84 (2H, d), 7.59 (2H, d), 7.37 (4H, m), 7.27 (1H, t), 4.65 (1H, s), 3.67 (3H, m), 2.27 (2H, m), 2.18 (1H, m), 2.08 (1H, m), 1.80 (1H, m), 1.69 (6H, m); LCMS m/z: 471 (M⁺+1).

The starting amine is prepared according to Alberts, Alfred W.; et al. Oxadiazolyl azabicyclooctane derivatives as squalene synthetase inhibitors for treatment of hypercholesterolemia. U.S. (1992), U.S. Pat. No. 5,135,935 and Baker, Raymond; et al. Preparation of oxadiazoles as central muscarinic acetylcholine receptor stimulants and pharmaceutical compositions containing them. Eur. Pat. Appl. (1989), EP 323864 A2.

### Example 5

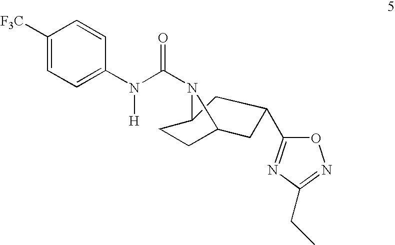

5: ¹H NMR (Acetone-d₆, 500 MHz): δ 8.53 (1H, s), 7.83 (2H, d), 7.59 (2H, d), 4.76 (1H, d), 4.58 (1H, m), 3.57 (1H, m), 2.68 (2H, q), 1.80-2.20 (7H, m), 1.70 (1H, m), 1.30 (3H, t); LCMS m/z: 395 (M⁺+1).

The starting secondary amine is prepared according to Fitch, Richard W.; et Homoepiboxidines further potent agonists for nicotinic receptors. Bioorganic & Medicinal Chemistry 2004, 12, 179-190.

### Example 6

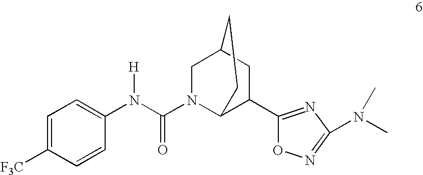

6: ¹H NMR (Acetone-d₆, 500 MHz): δ 8.07 (1H, s), 7.85 (2H, d), 7.59 (2H, d), 4.63 (1H, d), 3.68 (2H, m), 3.57 (1H, m), 3.13 (6H, s), 2.23 (3H, m), 1.80 (4H, m); LCMS m/z: 410 (M⁺+1).

The starting secondary amine is prepared according to Saunders, John; et al. Novel quinuclidine-based ligands for the muscarinic cholinergic receptor. Journal of Medicinal Chemistry 1990, 33, 1128-38.

### Example 7

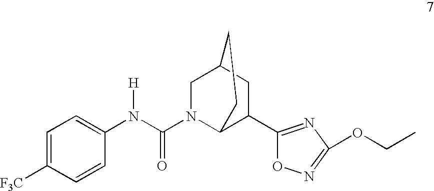

7: ¹H NMR (Acetone-d₆, 500 MHz): δ 8.08 (1H, s), 7.85 (2H, d), 7.59 (2H, d), 4.64 (1H, s), 4.36 (2H, q), 3.64 (3H, m), 2.21 (3H, m), 1.85 (1H, m), 1.75 (3H, m), 1.45 (3H, t); LCMS m/z: 411 (M⁺+1).

The starting secondary amine is prepared according to Saunders, John; et al. Novel quinuclidine-based ligands for the muscarinic cholinergic receptor. Journal of Medicinal Chemistry 1990, 33, 1128-38.

### Example 8

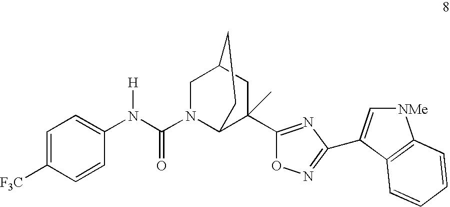

8: ¹H NMR (Acetone-d₆, 500 MHz): δ 8.19 (1H, d), 8.06 (1H, s), 7.89 (2H, d), 7.61 (2H, d), 7.56 (1H, d), 7.35 (1H, t), 7.29 (1H, t), 4.65 (1H, bs), 4.01 (3H, s), 3.70 (2H, m), 2.95 (1H, m), 2.13 (1H, m), 2.07 (2H, m), 1.90 (1H, m), 1.79 (1H, m), 1.62 (1H, m), 1.55 (3H, m); LCMS m/z: 510 (M⁺+1).

### Example 9

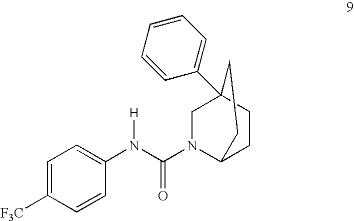

9: ¹H NMR (Acetone-d₆, 500 MHz): δ 8.01 (1H, s), 7.83 (2H, d), 7.55 (2H, d), 7.45 (2H, dd), 7.35 (2H, m), 7.27 (1H, t), 3.73 (2H, s), 2.06 (7H, m), 1.88 (2H, m); LCMS m/z: 375 (M⁺+1).

The starting secondary amine for the synthesis of 9 is prepared in accordance with Takeda, Mikio; et al. Azabicycloalkanes as analgetics. V. 4-Phenyl-2-azabicyclo[2,2,2]octanes. Chemical & Pharmaceutical Bulletin, 1977, 25, 775-83.

### Example 10

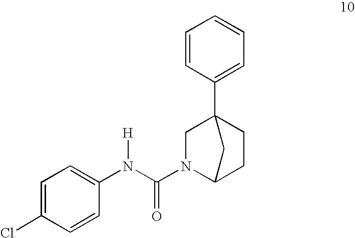

10: ¹H NMR (Acetone-d₆, 500 MHz): δ 7.72 (1H, bs), 7.64 (2H, m), 7.40 (4H, m), 7.26 (3H, m), 4.55 (1H, s), 3.62 (1H, dd), 3.49 (1H, dd), 2.09 (2H, m), 1.95 (4H, m); LCMS m/z: 327 (M⁺+1).

The starting secondary amines in Examples 11 and 12 are prepared in accordance with Huang, Xiaoqin; et al. Modeling Subtype-Selective Agonists Binding with α4β 2 and α7 Nicotinic Acetylcholine Receptors: Effects of Local Binding and Long-Range Electrostatic Interactions. Journal of Medicinal Chemistry 2006, 49, 7661-7674 and Csaba, Szantay; et al. Epi-epibatidine derivatives, a process and intermediates for preparing them and epi-epibatidine and medicaments containing the epi-epibatidine derivatives and/or epi-epibatidine and the use of them. Eur. Pat. Appl. (1995) EP 657455 A1.

### Example 11

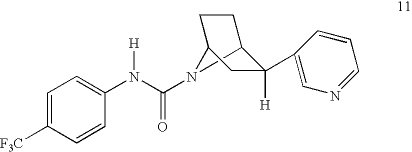

11: ¹H NMR (Acetone-d₆, 500 MHz): δ 8.56 (1H, d), 8.46 (1H, d), 8.15 (1H, s), 7.82 (2H, d), 7.73 (2H, d), 7.59 (2H, d), 7.36 (1H, m), 4.64 (1H, t), 4.57 (1H, t), 3.62 (1H, m), 2.37 (1H, m), 1.84 (2H, m), 1.63 (2H, m), 1.48 (1H, m); LCMS m/z: 362 (M⁺+1).

### Example 12

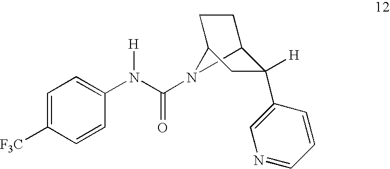

12: ¹H NMR (Acetone-d₆, 500 MHz): δ 8.53 (1H, d), 8.38 (1H, dd), 8.12 (1H, s), 7.73 (4H, m), 7.53 (2H, d), 7.24 (1H, m), 4.63 (1H, m), 4.41 (1H, d), 3.07 (1H, m), 2.08 (1H, m), 1.93 (3H, m), 1.75 (1H, m), 1.64 (1H, m); LCMS m/z: 362 (M⁺+1).

The starting secondary amines in Examples 11 and 12 are prepared in accordance with Carroll, F; et al. Synthesis, Nicotinic Acetylcholine Receptor Binding, and Antinociceptive Properties of 3′-Substituted Deschloroepibatidine Analogues. Novel Nicotinic Antagonists. Journal of Medicinal Chemistry 2005, 48, 1221.

### Example 13

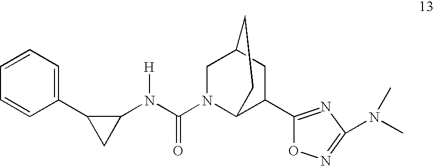

13: ¹H NMR (Acetone-d₆, 500 MHz): δ 7.23 (2H, m), 7.16 (3H, m), 6.05 (1H, s), 4.48 (1H, s), 119 (2H, m), 3.31 (6H, s), 2.95 (3H, s), 2.80 (1H, bs), 2.00-2.20 (3H, m), 1.60-1.75 (3H, m), 1.08-1.19 (2H, m); LCMS m/z: 382.6 (M⁺+1).

### Example 14

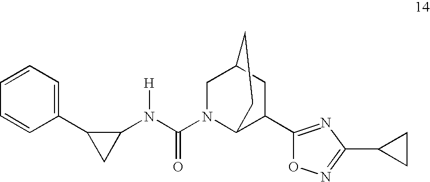

14: ¹H NMR (Acetone-d₆, 500 MHz): δ 7.23 (2H, m), 7.16 (3H, m), 6.03 (1H, s), 4.49 (1H, s), 3.48 (1H, m), 3.40 (2H, m), 2.80 (1H, bs), 2.15 (1H, m), 1.95-2.10 (4H, m), 1.50-1.75 (4H, m), 1.21 (1H, m), 1.15 (1H, m), 1.10 (1H, m), 1.03 (1H, m), 0.93 (2H, m); LCMS m/z: 379.7 (M⁺+1).

### Example 15

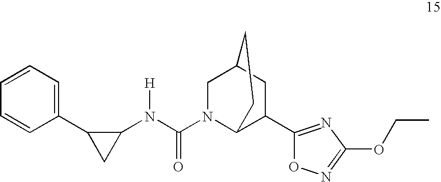

15: ¹H NMR (Acetone-d₆, 500 MHz): δ 7.23 (2H, m), 7.16 (3H, m), 6.04 (1H, s), 4.49 (1H, s), 4.31 (2H, m), 3.40 (4H, m), 2.81 (1H, bs), 2.05 (1H, bs), 2.04 (1H, bs), 2.00 (1H, bs), 1.70 (5H, m), 1.20 (2H, m), 1.18 (1H, m), 1.09 (1H, m); LCMS m/z: 383.6 (M⁺+1).

### Example 16

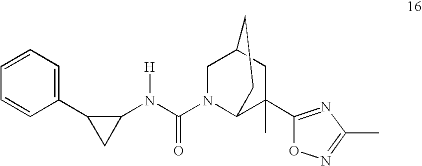

16: ¹H NMR (Acetone-d₆, 500 MHz): δ 7.23 (2H, m), 7.16 (3H, m), 6.04 (1H, d), 4.45 (1H, d), 3.38 (2H, m), 2.82 (1H, bs), 2.70 (1H, d), 2.32 (3H, d), 2.00-2.10 (2H, m), 1.72 (1H, m), 1.58 (3H, m), 1.34 (3H, d), 1.32 (1H, m), 1.08 (1H, m), 1.05 (1H, m); LCMS m/z: 367.6 (M⁺+1).

### Example 17

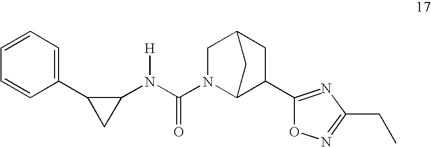

17: ¹H NMR (Acetone-d₆, 500 MHz): δ 7.23 (2H, m), 7.15 (3H, m), 6.07 (1H, s), 4.49 (1H, s), 3.39 (1H, bs), 3.32 (1H, bs), 2.71 (1H, bs), 2.67 (3H, m), 2.05 (4H, m), 1.75 (1H, d), 1.65 (1H, d), 1.25 (2H, m), 1.15 (1H, m), 1.09 (1H, m); LCMS m/z: 353 (M⁺+1).

The procedure described in the reference for the starting material of Example 2 can be used to prepare the starting secondary amine.

### Example 18

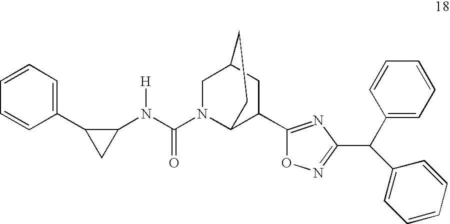

18: ¹H NMR (Acetone-d₆, 500 MHz): δ 7.39 (4H, m), 7.33 (4H, m), 7.25 (4H, m), 7.16 (3H, m), 6.05 (1H, s), 5.67 (1H, s), 4.53 (1H, s), 3.59 (1H, bs), 3.41 (1H, bd), 2.81 (1H, m), 2.22 (1H, m), 2.18 (1H, m), 2.08 (1H, bs), 2.05 (1H, bs), 2.00 (1H, m), 1.55-1.75 (4H, m), 1.18 (1H, m), 1.09 (1H, m); LCMS m/z: 505.6 (M⁺+1).

### Example 19

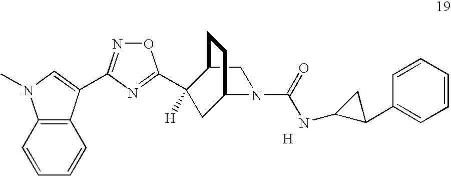

19: ¹H NMR (Acetone-d₆, 500 MHz): δ 8.25 (1H, d), 7.84 (1H, s), 7.19-7.41 (5H, m), 7.21 (3H, m), 5.85 (1H, s), 4.67 (1H, s), 3.88 (3H, s), 3.63 (1H, m), 3.41 (2H, bs), 2.94 (1H, m), 2.42 (1H, d), 2.20 (2H, m), 2.12 (1H, m), 1.82 (2H, m), 1.68 (1H, m), 1.25 (2H, m); LCMS m/z: 468.6 (M⁺+1).

The procedure described in the reference for the starting material of Example 2 can be used to provide the starting secondary amine.

### Example 20

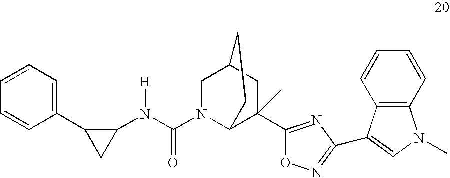

20: ¹H NMR (Acetone-d₆, 500 MHz): δ 8.15 (1H, t), 8.00 (1H, d), 7.51 (1H, t), 7.30 (1H, m), 7.25 (2H, m), 7.15 (3H, m), 6.09 (1H, d), 4.53 (1H, bs), 3.95 (3H, d), 2.82 (2H, m), 2.14 (1H, m), 2.02 (3H, m), 1.74 (1H, m), 1.68 (3H, m), 1.40 (4H, m), 1.20 (1H, m), 1.12 (1H, m); LCMS m/z: 482.7 (M⁺+1).

### DHET Production Assay

HEK293 (human kidney) cells were seeded at 4.2×10⁴ cells/well (100 ul) in 96-well plate in DMEM medium (high glucose) containing 10% FBS, 100 units/ml Penicillin and 100 ug/ml Streptomycin at 37° C. in a humidified atmosphere of 10% CO₂. After 24 h, the medium was changed to the same medium but without PBS for 1 h. The compound, diluted in DMSO, was added to each well for 1 h. Then, the substrate EET (3 uM final conc.) was added to each well for 2 h. At the end of the incubation period, 80 ul of medium was transferred to a new 96 well plate followed by LC-MS/MS analysis for the production of DHET.

### sEH Human Enzyme Assay

### Preparation of Recombinant sEH Human Enzyme

The DNA for expressing sEH was designed based on a rhesus monkey sEH cDNA, modified to optimize for expression both in E. coli and insect cells. The designed DNA fragment encodes a protein sequence that is identical to full length human sEH, and the DNA was synthesized in vitro. The DNA was then subcloned into the pET100 vector that will generate a fusion protein with an N-terminal His-tag. The recombinant protein was expressed in E. coli. The sEH enzyme was affinity purified by a Ni⁺⁺ column. His-tag was removed by Enter Kinase (EK) digestion. The purified enzyme aliquots were frozen and held at −80° C. for later use.

### Fluorescence Based Enzyme In Vitro Assay,

For each assay (100 ul), an aliquot of enzymes (about 1 nM final concentration) was incubated with a fluorescence substrate, S7 (10 uM final concentration), in sEH assay buffer (25 mM HEPES, pH7.0, 0.1 mg/ml BSA) in a 96-well plate. The kinetic reaction reading (Ex330/Em465) was conducted using a plate reader, Spectra max (Molecular Devices) at 25° C.

TABLE 2
IC₅₀ [nM] for human sEH enzyme inhibition

| Compound | IC₅₀ |
|---|---|
| 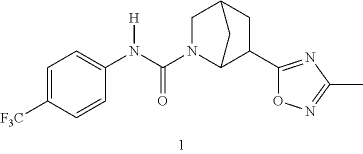 | 7 |
| 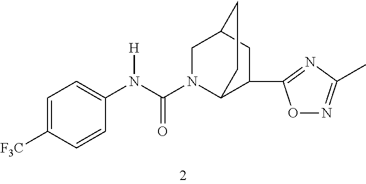 | 2 |
| 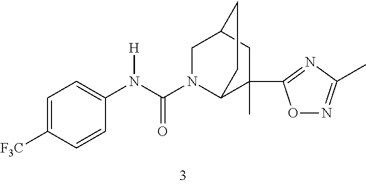 | 2 |
| 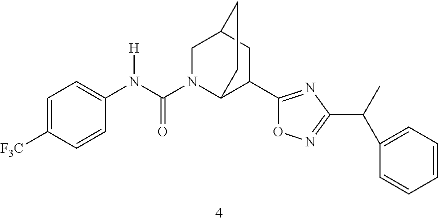 | 8 |
| 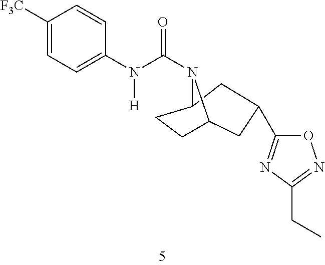 | 2 |
| 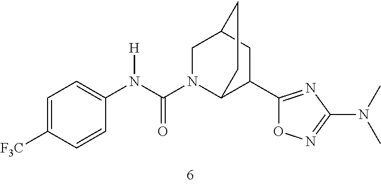 | 1 |
| 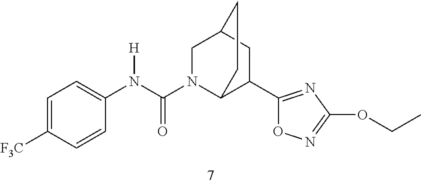 | 2 |
| 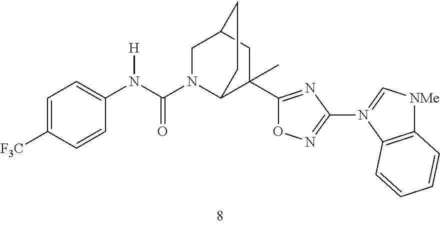 | 2 |
| 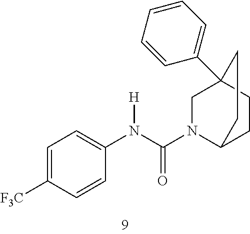 | 1 |
| 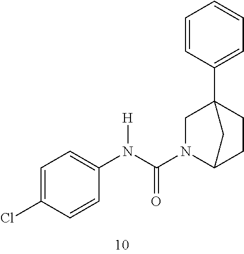 | 1 |
| 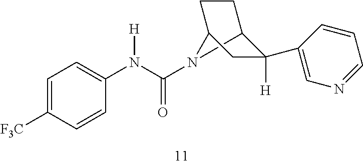 | 11 |
| 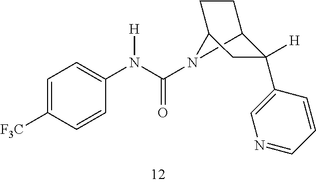 | 41 |
| 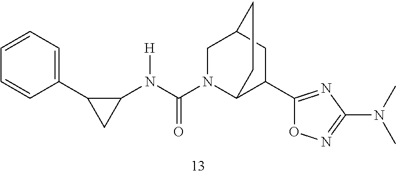 | 51 |
| 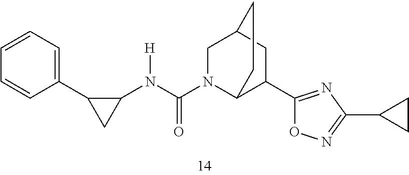 | 18 |
| 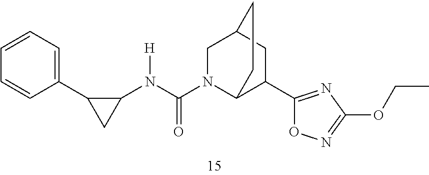 | 14 |
| 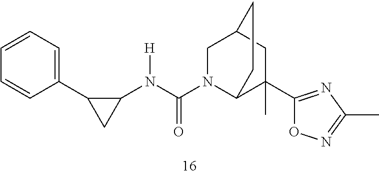 | 30 |
| 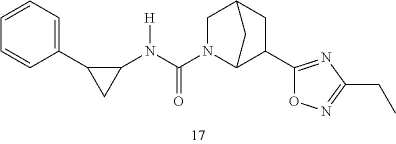 | 48 |
| 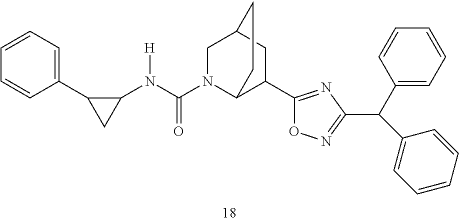 | 6 |
| 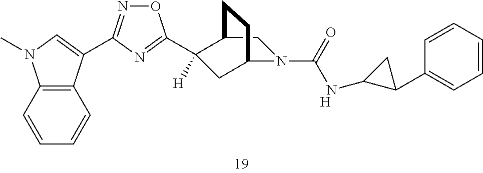 | 4 |
| 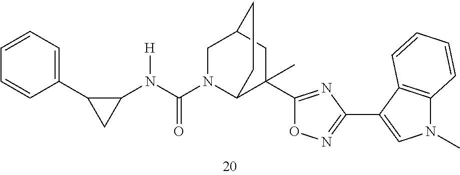 | 8 |

### Study Protocol: Oral Dosing of Enzyme Inhibitor for 14 Days in Male ZDF Rats

Seven weeks old, male ZDF rats (purchased from Charles River Labs) are conditioned with vehicle (0.5% methocel) for one week before the study is initiated. One week later, animals are prescreened and divided into 5 groups (n=8) based on the average baseline blood glucose levels and body weight. All the rats are then orally dosed (at a volume of 5 ml/kg) once daily with vehicle, inhibitor or rosiglitazone for continuous 14 days. The animals are fed Purina diet 5008 (ad lib.) throughout the study. Body weight and food intake are monitored twice a week. Ambient (fed) blood glucose levels are measured by glucometer (by tail clip) on day 1, 7 and 10 in the morning before dosing. On day 13, the animals treated with Vehicle 1, Inhibitor and rosiglitazone (n=6/group) are subjected to an oral glucose tolerance test (glucose: 2 g/kg) following overnight fast and one hr after receiving vehicle or compound. Blood glucose levels are measured at −60 (prior to compound dosing), 0 (prior to oral glucose), 20, 40, 60 and 120 min after glucose challenge. On day 14, two hours after the last dose, (fed) blood samples are collected from all the animals through cardiac puncture under CO₂ euthanasia. Whole blood target engagement, drug levels, plasma glucose, insulin, lipids, cytokines and other hormones are determined. Liver, skeletal muscle (Gastrocnemius), epididymal white adipose tissues and kidneys (cut longitudinally) from some animals are collected, wrapped into foils and frozen into liquid nitrogen immediately. Tissue target engagement, biomarkers and drug levels are measured.

Formulation: Vehicle 1: 0.5% methocel (with 10% vol. of cone. HCl and 20% vol. of 5N NaOH, pH˜7)
Inhibitor Compound: dissolve the compound in 10% volume of cone HCl (˜10N), add 70% volume of 0.5% methocel (at pH 3), mix well and then add 20% volume of 5N NaOH and mix. Adjust pH to neutral (pH 7). Vehicle 2: 0.5% methocel Rosiglitazone: in 0.5% methocel.

All patents, patent applications and publications that are cited herein are hereby incorporated by reference in their entirety. While certain preferred embodiments have been described herein in detail, numerous alternative embodiments are seen as falling within the scope of the invention.

1. A compound represented by formula I: 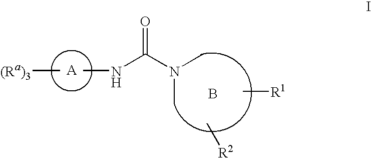
or a pharmaceutically acceptable salt thereof wherein:
ring A represents Aryl, HAR, Hetcy, C₃₋₇cycloalkyl, C₅₋₇cycloalkyl fused to an Aryl or HAR group, Aryl or HAR fused to C₅₋₇cycloalkyl, or C₆₋₁₀bicycloalkyl;
ring B represents a bridged bicyclic heterocyclic group having 1 nitrogen atom, 0-1 oxygen atom and 7-9 total atoms;
each R^{a} is defined as follows:
a) each R^{a} is H or halo, or
b) 1-2 R^{a} groups represent H or halo,
0-1 R^{a} represents Aryl, HAR or Hetcy, each of which being optionally substituted with 1-3 halo, C₁₋₃alkyl, haloC₁₋₃alkyl, OC₁₋₃alkyl or OhaloC₁₋₃alkyl groups, and 0-1 —CO₂R^{b} group;
and any remaining R^{a} groups are selected from the group consisting of: C₁₋₃alkyl, OC₁₋₃alkyl, haloC₁₋₃alkyl, OhaloC₁₋₃alkyl, S(O)ₓC₁₋₃alkyl, S(O)ₓ-haloC₁₋₃alkyl, S(O)ₓAryl wherein x is 0, 1 or 2, CO₂R^{b} or C₁₋₃alkyl-CO₂R^{b}, wherein R^{b} is H, C₁₋₄alkyl, haloC₁₋₄alkyl, Aryl, HAR or Hetcy;
R¹ is selected from the group consisting of: H, halo, C₁₋₃alkyl and haloC₁₋₃alkyl;
and R² is selected from Aryl(R^{x})ₚ and HAR(R^{x})_{q}, wherein p represents an integer of 1-5, q represents an integer of 1-4, each R^{x} is H, or 1-2 R^{x} groups are selected from the group consisting of: halo; C₁₋₅alkyl(R^{b})₃; OC₁₋₃alkyl(R^{b})₃; S(O)ₓC₁₋₃alkyl(R^{b})₃; S(O)ₓAryl; NH₂; NH(C₁₋₄alkyl(R^{b})₃); N(C₁₋₄alkyl(R^{b})₃)₂; CO₂R^{b}; Aryl, HAR and Hetcy, wherein said Aryl, HAR and Hetcy are each optionally substituted with 1-3 halo, C₁₋₃alkyl, haloC₁₋₃alkyl, OC₁₋₃alkyl or OhaloC₁₋₃alkyl groups, and 0-1 CO₂—C₁₋₆alkyl groups, and any remaining R^{x} groups are H, halo, C₁₋₃alkyl or haloC₁₋₃alkyl.

2. A compound in accordance with claim 1 wherein R^{a} is selected from the group consisting of H, F, Cl, C₁₋₃alkyl, OC₁₋₃alkyl, haloC₁₋₃alkyl, OhaloC₁₋₃alkyl and Aryl, HAR or Hetcy, each of which is optionally substituted with 1-3 halo, C₁₋₃alkyl, haloC₁₋₃alkyl, OC₁₋₃alkyl or OhaloC₁₋₃alkyl groups, and 0-1 —CO₂R^{b} group.

3. A compound in accordance with claim 1 wherein R^{a} is selected from the group consisting of: H, Cl, F, CH₃, CF₃, OCF₃ and Aryl that is optionally substituted with 1-3 halo, C₁₋₃alkyl, haloC₁₋₃alkyl, OC₁₋₃alkyl or OhaloC₁₋₃alkyl groups, and 0-1 —CO₂R^{b} group.

4. A compound in accordance with claim 1 wherein R^{a} is selected from the group consisting of: H, Phenyl, C₁ and CF₃.

5. A compound in accordance with claim 1 wherein ring A represents a member selected from the group consisting of: Aryl, HAR, C₃₋₇cycloalkyl, C₅₋₇cycloalkyl fused to an Aryl or HAR group, and Aryl or HAR fused to a C₅₋₇cycloalkyl group.

6. A compound in accordance with claim 1 wherein ring A represents Aryl, HAR or C₃₋₇cycloalkyl.

7. A compound in accordance with claim 1 wherein ring A represents a member selected from the group consisting of: phenyl, a 5-10 membered heteroaryl group selected from the group consisting of pyridyl, pyrimidyl, pyrazolyl and thienyl and a C₃₋₅cycloalkyl group.

8. A compound in accordance with claim 1 wherein ring A represents a member selected from the group consisting of phenyl, pyridyl and a C₃₋₅cycloalkyl group.

9. A compound in accordance with claim 1 wherein ring A represents a phenyl or cyclopropyl ring.

10. A compound in accordance with claim 1 wherein ring A represents cyclopropyl.

11. A compound in accordance with claim 1 wherein ring B represents a 7-8 membered bicyclic heterocyclic group containing one nitrogen atom.

12. A compound in accordance with claim 1 wherein ring B represents a member selected from the group consisting of:

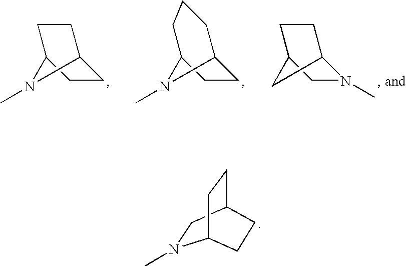

13. A compound in accordance with claim 1 wherein R¹ is selected from the group consisting of H and CH₃.

14. A compound in accordance with claim 1 wherein R² is selected from the group consisting of: Aryl(R^{x})ₚ in which the Aryl portion represents phenyl, p is an integer of 1-5, and each R^{x} is hydrogen, or 1-2 R^{x} groups represent halo, C₁₋₃allyl, haloC₁₋₃alkyl, OC₁₋₃alkyl or haloOC₁₋₃alkyl, and any remaining R^{x} groups represent hydrogen.

15. A compound in accordance with claim 1 wherein R² represents Aryl, which is phenyl, and all R^{x} groups represent hydrogen.

16. A compound in accordance with claim 1 wherein R² represents HAR(R^{x})_{q}, q is an integer of from 1-4, HAR represents a 5-6 membered heteroaryl ring with one nitrogen atom, 0-1 oxygen or sulfur atom, and 0-2 additional nitrogen atoms, and each R^{x} group represents hydrogen, or 1-2 R^{x} groups represent a member selected from the group consisting of: methyl, ethyl, cyclopropyl, methylamino, dimethylamino, methoxy, ethoxy, 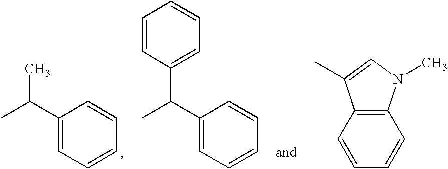
and any remaining R^{x} groups represent hydrogen.

17. A compound in accordance with claim 1 wherein:
R² represents HAR(R^{x})_{q},
q represents an integer from 1-4;
HAR is selected from the group consisting of: pyridyl and oxadiazolyl, and
the R^{x} groups represent hydrogen, or 1-2 R^{x} groups represent a member selected from the group consisting of: methyl, ethyl, cyclopropyl, methylamino, dimethylamino, methoxy, ethoxy, 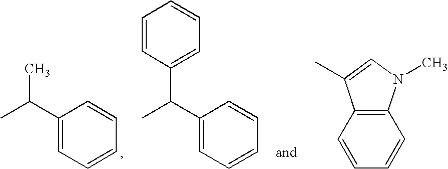
and any remaining R^{x} groups represent hydrogen.

18. (canceled)

19. A compound in accordance with claim 1 represented by formula I: 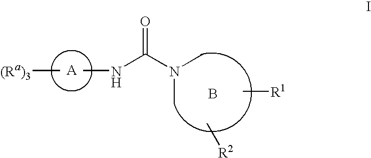
or a pharmaceutically acceptable salt or solvate thereof wherein:
ring A represents Aryl, HAR or C₃₋₇cycloalkyl;
ring B represents a 7-8 membered bicyclic heterocyclic group containing one nitrogen atom;
R^{a} is selected from the group consisting of: H, Cl, F, CH₃, CF₃, OCF₃ and Aryl that is optionally substituted with 1-3 halo, C₁₋₃alkyl, haloC₁₋₃alkyl, OC₁₋₃alkyl or OhaloC₁₋₃alkyl groups, and 0-1 —CO₂R^{b} group;
R¹ is selected from the group consisting of H and CH₃;
R² is selected from the group consisting of:
Aryl(R^{x})ₚ in which the Aryl portion represents phenyl, p is an integer of 1-5, and each R^{x} is hydrogen, or 1-2 R^{x} groups represent halo, C₁₋₃alkyl, haloC₁₋₃alkyl, OC₁₋₃alkyl and haloOC₁₋₃alkyl and any remaining R^{x} groups represent hydrogen, and
HAR(R^{x})_{q}, wherein q is an integer of from 1-4, HAR represents a 5-6 membered heteroaryl ring with one nitrogen atom, 0-1 oxygen or sulfur atom, and 0-2 additional nitrogen atoms, and each R^{x} group represents hydrogen, or 1-2 R^{x} groups represent a member selected from the group consisting of: methyl, ethyl, cyclopropyl, methylamino, dimethylamino, methoxy, ethoxy, 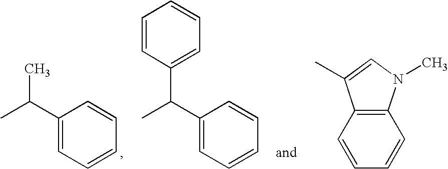
and any remaining R^{x} groups represent hydrogen.

20. A compound in accordance with claim 1 selected from Table 1:
TABLE 1 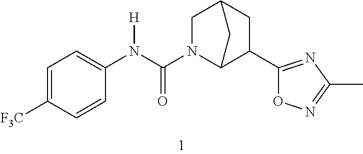 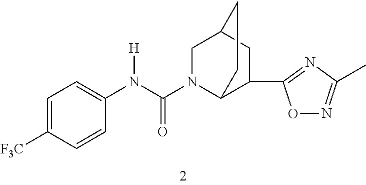 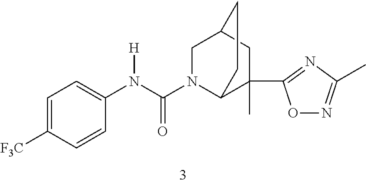 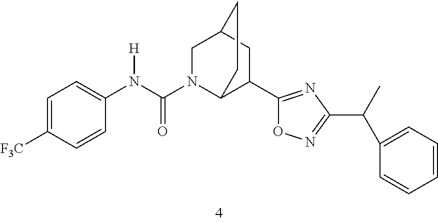 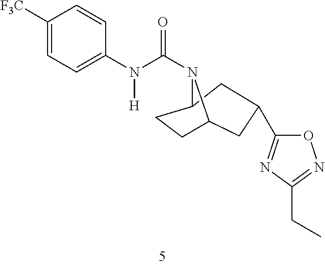 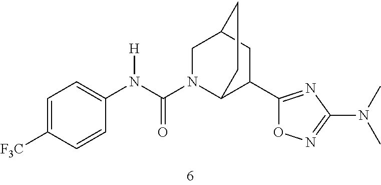 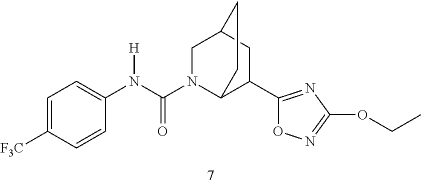 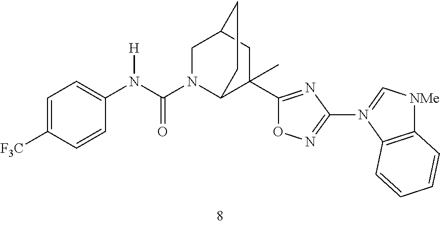 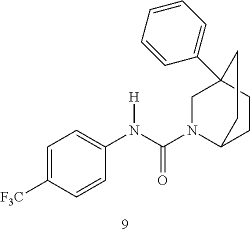 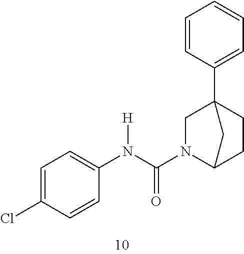 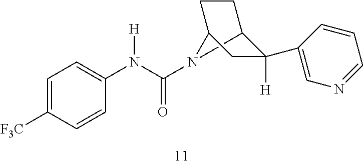 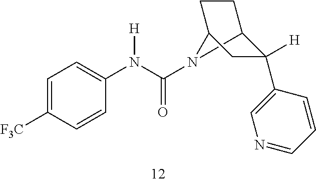 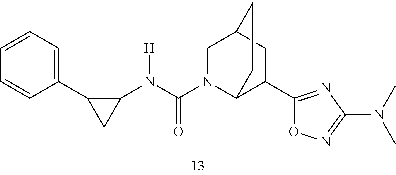 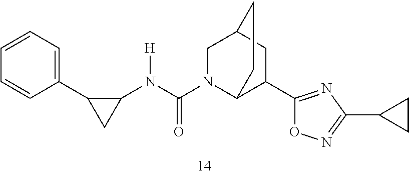 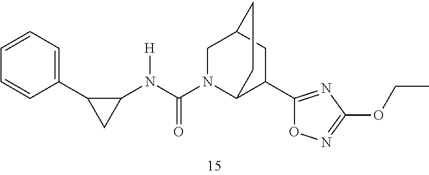 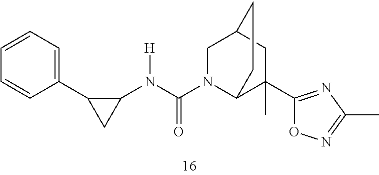 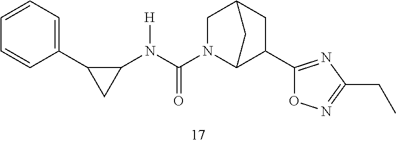 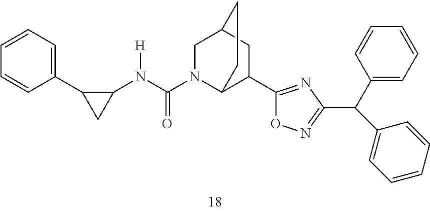 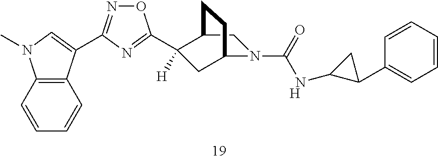 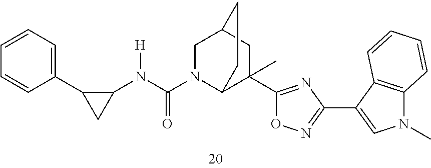
and the pharmaceutically acceptable salts thereof.

21. A pharmaceutical composition comprised of a compound in accordance with claim 1 in combination with a pharmaceutically acceptable carrier.

22. A method of treating diabetes in a mammalian patient in need of such treatment comprising administering to the patient a compound in accordance with claim 1 in an amount that is effective for treating diabetes.

23-25. (canceled)
